SEQUENCE LISTING (5 sequences)

SEQ ID NO: 1 - RNA, 7405 nt, Coxsackievirus A 21
uuaaaacagcucugggguuguucccaccccagaggcccacguggcggcuaguacucugguauuacgguaccuuuguacgccuguuuuguaucccuucccccguaacuuuagaagcuuaucaaagguucaauagcagggguacaaaccaguaccucuacgaacaagcacuucuguuuccccggugauaucacauagacuguacccacggucaaaagugauugauccguuauccgcuugaguacuucgagaagccuaguaucaccuuggaaucuucgaugcguugcgcucaacacucugccccgaguguagcuuaggcugaugagucugggcacuccccaccggcgacgguggcccaggcugcguuggcggccuacccauggcugaugccgugggacgcuaguugugaacaaggugugaagagccuauugagcuacucaagaguccuccggccccugaaugcggcuaauccuaaccacggagcaaccgcucacaacccagugaguagguugucguaaugcguaagucuguggcggaaccgacuacuuuggguguccguguuucccuuuauauucauacuggcugcuuauggugacaauuuacaaauuguuaccauauagcuauuggauuggccacccaguauugugcaauauauuugaguguuucuuucauaagccuuauuaacaucacauuuuuaaucacaauaaacagugcaaaugggggcucaaguuucaacgcaaaagaccggugcgcacgagaaucaaaacguggcagccaauggauccaccauuaauuacacuacuaucaacuauuacaaagacagugcgaguaauuccgcuacuagacaagaccucucccaagauccaucaaaauucacagaaccgguuaaggacuuaauguugaaaacagcaccagcucuaaacucgccuaacguggaagcauguggguacagugaccgugugaggcaaaucacuuuaggcaacucgacuauuacuacacaagaagcagccaaugcuauuguugcuuacggugaauggcccacuuacauaaaugauucagaagcuaauccgguagaugcacccacugagccagauguuaguagcaaccgguuuuacacccuagaaucggugucuuggaagaccacuucaaggggauggugguggaaguuaccagauuguuugaaggacaugggaauguuuggucagaauauguacuaucacuacuuggggcgcucugguuacaccauucauguccagugcaacgcuucaaaauuucaccaaggggcguuaggaguuuuucugauaccagaguuugucauggcuugcaacacugagaguaaaacgucauacguuucauacaucaaugcaaauccuggugagagaggcggugaguuuacgaacaccuacaauccguuaaauacagacgccagugagggcagaaaguuugcagcauuggauuauuugcuggguucugguguucuagcaggaaacgccuuuguguacccgcaccagaucaucaaccuacguaccaacaacagugcaacaauuguggugccauacguaaacucacuugugauugauuguauggcaaaacacaauaacuggggcauugucauauuaccacuggcacccuuggccuuugccgcaacaucgucaccacaggugccuauuacagugaccauugcacccauguguacagaauucaauggguugagaaacaucaccgucccaguacaucaaggguugccgacaaugaacacaccugguuccaaucaauuccuuacaucugaugacuuccagucgcccugugccuuaccuaauuuugauguuacuccaccaauacacauacccggggaaguaaagaauaugauggaacuagcugaaauugacacauugaucccaaugaacgcaguggacgggaaggugaacacaauggagauguaucaaauaccauugaaugacaauuugagcaaggcaccuauauucuguuuaucccuaucaccugcuucugauaaacgacugagccgcaccauguugggugaaauccuaaauuauuacacccauuggacgggguccaucagguucaccuuucuauuuugugguaguaugauggccacugguaaacugcuccucagcuauuccccaccgggagcuaaaccaccaaccaaucgcaaggaugcaaugcuaggcacacacaucaucugggaccuaggguuacaauccaguuguuccaugguugcaccguggaucuccaacacaguguacagacggugugcacgugaugacuucacugagggcggauuuauaacuugcuucuaucaaacuagaauugugguaccugcuucaaccccuaccaguauguucauguuaggcuuuguuagugcguguccagacuucagugucagacugcuuagggacacuccccauauuagucaaucgaaacuaauaggacguacacaaggcauugaagaccucauugacacagcgauaaagaaugccuuaagagugucccaaccacccucgacccagucaacugaagcaacuaguggagugaauagccaggaggugccagcucuaacugcuguggaaacaggagcaucuggucaagcaauccccagugaugugguggaaacuaggcacgugguaaauuacaaaaccaggucugaaucgugucuugagucauucuuugggagagcugcgugugucacaauccuauccuugaccaacuccuccaagagcggagaggagaaaaagcauuucaacauauggaauauuacauacaccgacacuguccaguuacgcagaaaauuagaguuuuucacguauuccagguuugaucuugaaaugacuuuuguauucacagagaacuauccuaguacagccaguggagaagugcgaaaccagguguaccagaucauguauauuccaccaggggcaccccgcccaucauccugggaugacuacacauggcaauccucuucaaacccuuccaucuucuacauguauggaaaugcaccuccacggaugucaauuccuuacguagggauugccaaugccuauucacacuucuacgauggcuuugcacgggugccacuugagggugagaacaccgaugcuggcgacacguuuuacgguuuaguguccauaaaugauuuuggaguuuuagcaguuagagcaguaaaccgcaguaauccacauacaauacacacaucugugagaguguacaugaaaccaaaacacauucgguguuggugccccagaccuccucgagcuguauuauacaggggagagggaguggacaugauauccagugcaauucuaccucugaccaagguagacucaauuaccacuuuuggguuuggucaucagaacaaagcaguguacguugccgguuacaagauuugcaacuaccaccuagcaaccccaagugaucacuugaaugcaauuaguauguuaugggacagggauuuaauggugguggaaucuagagcccagggaacugauaccaucgccagauguaguugcagguguggaguuuacuauugugaaucuaggaggaaguacuacccugucacuuuuacuggcccaacguuucgauucauggaagcaaacgacuacuauccagcaagauaccagucucacaugcugauagggugcggauuugcagaacccggggacugcggugggauacugaggugcacucaugggguaauugguaucauuacugcaggaggugaagggguaguagccuuugcugacauuagagaccucuggguguaugaagaggaggccauggaacagggaauaacaagcuacaucgaaucucucggcacagccuuuggcgcaggguucacccacacaaucagugagaaagugacugaauugacaacaaugguuaccagcacuaucacagaaaaacuacugaaaaacuuggugaaaauagugucggcucuagugauuguugugagaaauuaugaggacacuaccacgauccuugcaacacuagcacuacucgggugugauauaucuccuuggcaaugguugaagaagaaggcaugugacuuacuagagauuccuuaugugaugcgccaaggugauggguggaugaagaaauucacagaggcgugcaaugcagcuaaaggcuuagaguggauuagcaacaaaauuuccaaguuuauagauugguugaaguguaaaauuaucccagacgcuaaggacaagguggaauuucucaccaaguugaaacagcuagacauguuggaaaaucaaauugcaaccauccaccaaucuugccccagccaagaacaacaagagauucuuuucaacaaugugagauggcuagcaguccagucccgucgguuugcaccauuauacgcuguggaggcacgccgaauuaacaaaauggagagcacaauaaacaauuauauacaguucaagagcaaacaccguauugaaccaguauguaugcucauucaugggucaccagggacggguaaaucuauagcuacuucauuaauagguagagcaauagcagagaaggaaagcacaucagucuauucaaugccaccugacccaucucacuuugauggcuauaaacaacaagggguagugauuauggacgaccuaaaccaaaaccccgaugguauggacaugaaacuguuuugccaaaugguaucaacaguggaguuuauuccuccaauggccucauuagaggagaagggcauuuuguuuacaucugauuauguccuggcuucuaccaacucucauucaauuguaccacccacaguggcucacagugaugccuuaaccagacgauuugcauuugauguggagguuuacacgaugucugaacauucagucaaaggcaaacugaauauggccacggccacucaauuguguaaggauuguccaacaccugcaaauuuuaaaaaguguugcccucucguuuguggaaaggccuugcaauuaauggacagguacaccagacaaagguucacuguagaugagauuaccacauuaaucaugaaugagaaaaacagaagggccaauaucggcaauugcauggaagccuuguuucaaggaccauuaagguauaaagauuugaagaucgaugugaagacaguuccccccccugagugcaucagugauuuguuacaagcaguggauucucaagagguuagggauuacugugagaagaaaggcuggaucguuaacguuacuagccagauucaacuagaaaggaacaucaauagggccaugacuauacuccaagcuguuaccacauucgcagcagucgcaggaguaguguauguaauguacaaacucuucgccggucaacagggugcauacacuggcuugccaaacaaaaaacccaaugucccuacuaucagagucgcuaaaguccaggggccaggauuugacuacgcaguggcaauggcaaaaagaaacauaguuacugcaaccaccaccaagggugaauuuaccaugcuaggggugcaugauaauguagcaauauugccaacccaugccgcuccaggagaaaccauuauuauugaugggaaagaaguagagauccuagaugccagagccuuagaagaucaagcgggaaccaaucuugagaucaccauuauuacucuaaaaagaaaugagaaguuuagagacaucagaucacauauucccacccaaauuacugaaacuaacgauggaguguugaucgugaacacuagcaaguaccccaauauguauguccccguuggugcugugaccgaacagggauaucuuaaucucaguggacgucaaacugcucgcacuuuaauguacaacuuuccaacaagggcaggccagugcggaggaaucaucacuuguacuggcaaagucauugggaugcauguuggcgggaacgguucacauggguuugcagcagcccucaagcgaucauacuucacucaaaaucagggcgaaauccaguggaugaggucaucaaaagaagugggguaccccauuauaaaugccccauccaagacaaaguuagaacccagugcuuuccacuauguuuuugaagguguuaaggaaccagcuguacucacuaagaaugaccccagacuaaaaacagauuuugaagaagccaucuuuucuaaauauguggggaacaaaauuacugaaguggacgaguacaugaaagaagcaguggaucacuaugcaggacaguuaaugucacuggauaucaacacagaacagaugugccuggaggaugccauguacggcaccgauggucuugaggcccuggaucuuagcacuagugcuggauauccuuauguugcaauggggaaaaagaaaagagacauucuaaauaaacagaccagagauacuaaggagaugcagagacuuuuagauaccuauggaaucaaucuaccauuagucacguacgugaaagaugaacucaggucaaagacuaaaguggaacaaggaaagucaagauugauugaagcuuccagccuuaaugauucaguugcaaugagaauggccuuuggcaaucuuuacgcagcuuuccacaagaauccagguguggugacaggaucagcaguugguugugacccagauuuguuuuggaguaagauaccagugcuaauggaagaaaaacucuucgcuuuugacuacacaggguaugaugccucacucagcccugcuugguuugaagcucuuaaaaugguguuagaaaaaauuggauuuggcaguagaguagacuauauagacuaccugaaccacucucaccaccuuuacaaaaacaagacuuauugugucaaaggcggcaugccauccggcugcucuggcaccucaauuuucaacucaaugauuaacaaccugaucauuaggacgcuuuuacugagaaccuacaagggcauagacuuggaccauuuaaaaaugauugccuauggugaugacgugauagcuuccuacccccaugagguugacgcuagucuccuagcccaaucaggaaaagacuauggacuaaccaugacuccagcagauaaaucaguaaccuuugaaacagucacaugggagaauguaacauuucugaaaagauuuuucagagcagaugagaaguauccauuccuggugcauccagugaugccaaugaaagaaauucacgaaucaaucagauggaccaaggacccuagaaacacacaggaucacguacgcucguugugccuauuagcuuggcacaacggugaagaagaauacaauaaauuuuuagcuaaaaucagaagugugccaauuggaagagcuuuauugcucccagaguacucuacauuguaccgccgauggcucgacucauuuuaguaacccuaccucagucggauuggauuggguuacacuguuguagggguaaauuuuucuuuaauucggag

SEQ ID NO: 2 - protein, 298 aa, Coxsackievirus A 21
GIEDLIDTAIKNALRVSQPPSTQSTEATSGVNSQEVPALTAVETGASGQAIPSDVVETRHVVNYKTRSESCLESFFGRAACVTILSLTNSSKSGEEKKHFNIWNITYTDTVQLRRKLEFFTYSRFDLEMTFVFTENYPSTASGEVRNQVYQIMYIPPGAPRPSSWDDYTWQSSSNPSIFYMYGNAPPRMSIPYVGIANAYSHFYDGFARVPLEGENTDAGDTFYGLVSINDFGVLAVRAVNRSNPHTIHTSVRVYMKPKHIRCWCPRPPRAVLYRGEGVDMISSAILPLTKVDSITTF

SEQ ID NO: 3 - protein, 272 aa, Coxsackievirus A 21
SPNVEACGYSDRVRQITLGNSTITTQEAANAIVAYGEWPTYINDSEANPVDAPTEPDVSSNRFYTLESVSWKTTSRGWWWKLPDCLKDMGMFGQNMYYHYLGRSGYTIHVQCNASKFHQGALGVFLIPEFVMACNTESKTSYVSYINANPGERGGEFTNTYNPLNTDASEGRKFAALDYLLGSGVLAGNAFVYPHQIINLRTNNSATIVVPYVNSLVIDCMAKHNNWGIVILPLAPLAFAATSSPQVPITVTIAPMCTEFNGLRNITVPVHQ

SEQ ID NO: 4 - protein, 240 aa, Coxsackievirus A 21
GLPTMNTPGSNQFLTSDDFQSPCALPNFDVTPPIHIPGEVKNMMELAEIDTLIPMNAVDGKVNTMEMYQIPLNDNLSKAPIFCLSLSPASDKRLSRTMLGEILNYYTHWTGSIRFTFLFCGSMMATGKLLLSYSPPGAKPPTNRKDAMLGTHIIWDLGLQSSCSMVAPWISNTVYRRCARDDFTEGGFITCFYQTRIVVPASTPTSMFMLGFVSACPDFSVRLLRDTPHISQSKLIGRTQ

SEQ ID NO: 5 - protein, 69 aa, Coxsackievirus A 21
MGAQVSTQKTGAHENQNVAANGSTINYTTINYYKDSASNSATRQDLSQDPSKFTEPVKDLMLKTAPALN

(19) United States
(12) Patent Application Publication
(10) Pub. No.: US 2021/0187049 A1
(43) Pub. Date: Jun. 24, 2021

(54) PURIFIED COMPOSITIONS OF ENTEROVIRUSES AND METHODS OF PURIFICATION WITH GLUTATHIONE AFFINITY CHROMATOGRAPHY

(71) Applicant: Merck Sharp & Dohme Corp., Rahway, NJ (US)

(72) Inventors: Erik M. Curtis, North Wales, PA (US); Spyridon Konstantinidis, Lansdale, PA (US); Murphy Poplyk, Hatfield, PA (US); Andrew Ryan Swartz, Chalfont, PA (US); Marc D. Wenger, Allentown, PA (US)

(73) Assignee: Merck Sharp & Dohme Corp., Rahway, NJ (US)

(21) Appl. No.: 17/125,002

(22) Filed: Dec. 17, 2020

Related U.S. Application Data
(60) Provisional application No. 62/951,078, filed on Dec. 20, 2019.

(51) Int. Cl. A61K 35/768; C12N 7/02

(52) U.S. Cl. CPC A61K 35/768; C12N 2770/32351; C12N 7/02

(57) ABSTRACT

The present invention relates to purified compositions of enteroviruses, pharmaceutical compositions thereof and a glutathione affinity chromatography process for the purification of enteroviruses.

### FIELD OF THE INVENTION

The present invention relates to purified compositions of enteroviruses and a glutathione affinity chromatography process for the purification of enteroviruses.

### REFERENCE TO SEQUENCE LISTING SUBMITTED ELECTRONICALLY

The sequence listing of the present application is submitted electronically via EFS-Web as an ASCII formatted sequence listing with a file name 24943USNP-SEQLIST-17NOV2020.txt, creation date of Nov. 17, 2020, and a size of 17.6 kb. This sequence listing submitted via EFS-Web is part of the specification and is herein incorporated by reference in its entirety.

### BACKGROUND OF THE INVENTION

The Enterovirus genus of the Picornaviridae family are small, non-enveloped, single stranded positive sense RNA viruses that contain several species of human pathogens including polioviruses, coxsackieviruses, echoviruses, numbered enteroviruses, and rhinoviruses [1]. Aside from the well-studied poliovirus, there has been an influx of research into the development of vaccines and therapeutics for diseases caused by non-polio enteroviruses such as EV-A71 (hand foot and mouth disease) [2], EV-D68 (respiratory disease) and Coxsackievirus A24 (acute hemorrhagic conjunctivitis) [3]. Enteroviruses have also been evaluated for use as oncolytic viral immunotherapies [4]. Coxsackievirus A21 (CVA21), derived from the wild-type strain, is currently being evaluated in phase 1b/2 clinical trials as a treatment for multiple types of cancer due to its selective infection and oncolysis of tumors overexpressing cell surface receptors ICAM-1[5].

The increasing demand for enterovirus viral vaccines and immunotherapies could challenge the conventional production platform. Gradient ultracentrifugation is commonly employed for the enrichment of full, genome containing capsids and impurity clearance, but may be a potential bottleneck in the purification process due to its low-throughput and labor-intensive protocols [6] (FIG. 1A). As evidenced by the recombinant adeno-associated viral gene therapy purification platform, a shift from gradient ultracentrifugation towards chromatography-based methods may improve scalability and productivity [7]. No chromatographic technique has been demonstrated for empty (lacking genome; product impurity) and full (genome containing; target product) enterovirus particle separation. There remains a need for a chromatography-based alternative to gradient ultracentrifugation that is capable of removing empty capsids and contaminating impurities to produce a purified composition of infectious, mature virions. This would enable an enterovirus purification process that is more suitable for large-scale commercial manufacturing.

### SUMMARY OF THE INVENTION

The present invention provides purified compositions of CVA21, wherein the genome to infectivity ratio is less than about 5000 genome/pfu; the particle to infectivity ratio is less than about 5000 particle/pfu; the VP0 to VP2 ratio is less than about 0.01 as measured by reverse phase HPLC or UPLC; or the total VP1+VP2+VP3+VP4 peak area/total peak area is at least 95% as measured by CE-SDS. The present invention also provides a pharmaceutical composition comprising the purified compositions described above. The present invention also provides a method of treating cancer by administering the pharmaceutical compositions of the invention. In another aspect, the present invention provides use of glutathione affinity chromatography to purify enterovirus from one or more impurities. In one embodiment, the method selectively captures and enriches genome-containing full mature enterovirus virions from infected host-cell culture harvests, thereby removing one or more impurities such as non-infectious genome-lacking enterovirus procapsids, host-cell proteins (HCP), host-cell DNA (HC-DNA), and media-related impurities such as bovine serum albumen (BSA). The present invention also comprises use of anion exchange chromatography to purify enterovirus from one or more impurities.

### BRIEF DESCRIPTION OF THE DRAWINGS

The foregoing and other objects, features and advantages of the invention will be apparent from the following more particular description of various embodiments of the invention, as illustrated in the accompanying drawings.

FIG. 1A-B: A: Description of gradient ultracentrifugation process. Clarified cell culture harvests are first concentrated for volume reduction. The gradient is prepared in an ultracentrifuge tube and the sample is loaded on top. After centrifugation, the gradient is fractionated, and selected fractions are pooled. The pooled fractions are dialyzed to remove the gradient solution. B: Description of GSH affinity chromatography process. Clarified cell culture harvest is directly loaded to the GSH affinity column. The column is washed to remove impurities and the purified virus is eluted. The column can be regenerated for future use.

FIG. 2: Enterovirus morphogenesis and assembly. Five protomers consisting of VP0+VP1+VP3 assemble to form a pentamer. Empty procapsids may be formed from the reversible assembly of free pentamers. After 12 pentamers condense and encapsidate the newly synthesized genome on a replication organelle to form a provirion, VP0 is autocatalytically cleaved to form VP4+VP2 and a mature virion is formed. Mature virions are the only particle containing VP4 and are capable of being infectious, but not all mature virions may be infectious. Mature virions may degrade into A-particles and empty capsids of A-particles. Adapted from [10].

FIG. 3: Example chromatography for GSH affinity chromatography chromatogram operation using the Akta Pure and analyzed by UNICORN software using CVA21. Absorbance at 280 nm (UV 1_280, solid line) trace in mAUs and conductivity (Cond, dashed line) trace in mS/cm for GSH affinity chromatography operation volume in mL. Top figure represents the full chromatogram. Bottom figure, depicted within the dotted box of top figure, represents the wash, elution and strip steps.

FIG. 4: Reducing 12% acrylamide Bis-Tris SDS-PAGE with silver stain of GSH affinity chromatography process using CVA21. Host cell and media impurities in the clarified harvest were cleared in the GSH flow-through (GSH FT) and Wash (GSH W1-2) steps. CVA21 was eluted at high concentration and purity (trace amounts of BSA detected) with only VP1-VP2-VP3 viral capsid bands observed (VP4 (7 kDa) runs off the gel) and minimal VP0. The GSH elution (GSH Elute) sample was compared with a gradient ultracentrifugation purified (UC Pure) CVA21.

FIG. 5: Relative comparison of CVA21 clarified harvest and GSH chromatography flow-through (GSH FT) and elution (GSH Elute) samples to ultracentrifugation purified material (UC Pure) using capillary electrophoresis quantitative western blot (Protein Simple). Total capsid particles detected by an anti-VP1 polyclonal antibody (pAb) and the VP0/VP4 signal ratio detected with an anti-VP4 pAb. Particles with high VP0/VP4 ratio (high amount of VP0 containing particles: empty procapsid, provirion, pentamer, protomer) flowed through while viral particles with low VP0/VP4 (high amount of VP4 containing particles: mature virion only) ratio were eluted from the GSH chromatography column. Compared to the UC Pure, the GSH elution resulted in a 10-fold lower VP0/VP4 ratio, indicating a high mature virus particle purity.

FIG. 6A-B: Sucrose gradient characterization of GSH affinity elution. 1 mL sample loaded to 15-42% w/v sucrose gradient and spun for 100 min at 230,000 g. 12×1 mL fractions taken and empty capsids (procapsid or degraded A-particle) expected in fractions 5-8 and full capsids (mature virion or provirion) expected in fractions 9-12. 6A: Reducing 12% acrylamide Bis-Tris SDS-PAGE with silver stain of a GSH affinity chromatography purified elution. Viral protein bands VP1-VP2-VP3 (VP4 not shown) detectable with no VP0 and no empty capsids detected. 6B: Total particle distribution in sucrose gradient fractions using capillary electrophoresis quantitative western blotting (Protein Simple) with an anti-VP1 polyclonal antibody. The GSH affinity chromatography elution contained a full mature virion population of about 99.8% of the total particles.

FIG. 7: BSA clearance across GSH affinity chromatography fractions detected by capillary electrophoresis quantitative western blot (Protein Simple) with an anti-BSA primary antibody. % BSA mass relative to clarified harvest sample calculated by the mass of BSA in the sample divided by the initial mass of BSA in the clarified harvest.

FIG. 8: Example chromatogram for GSH affinity chromatography chromatogram operation of Arm 9 (See Table 3) using the GSH Robocolumn and Tecan Freedom EVO 150. Absorbance at 280 nm (solid line) and estimated NaCl concentration (dotted line) traces shown. Top figure represents the full chromatogram. Bottom figure, depicted within the dotted box of top figure, represents the wash, elution and strip steps. Elution fraction sampled at ˜61-CV.

FIG. 9A-B: Reducing 12% acrylamide Bis-Tris SDS-PAGE with silver stain of GSH affinity chromatography purification of multiple enterovirus serotypes. Images of clarified cell culture harvests (FIG. 9A) and GSH elution fractions (FIG. 9B) for the evaluated enterovirus strains (1-9) and the mark are shown. Enterovirus capsid viral protein (VP) bands detectable in GSH elution for Echovirus 1 (1), Rhinovirus 1B (2), Rhinovirus 35 (3), Coxsackievirus A 13 (4), Coxsackievirus A 15 (5), Coxsackievirus A 18 (6), Coxsackievirus A 20b (7), and Coxsackievirus A 21 (8, 9).

FIG. 10: Reducing 12% acrylamide Bis-Tris SDS-PAGE with silver stain of GSH affinity chromatography of CVA21 produced with different upstream conditions. Clarified bulk (CB) prediluted 100× and GSH Elution (GSH) loaded neat samples shown for Experiment Arms 1-5. Viral protein (VP) bands in GSH elution samples identified as VP0, VP1, VP2, and VP3. Higher VP0 detected in Arms A, C, and D indicate differences in empty procapsid clearance across the GSH chromatography step.

FIG. 11: Comparison of the capillary electrophoresis quantitative western VP0/VP4 signal ratio detected with an anti-VP4 pAb for clarified harvest and GSH elution samples from Arms 1-5 relative to ultracentrifugation purified virus. Differences in empty procapsid/full mature virus particle ratio as estimated by VP0/VP4 ratio observed in the GSH elution samples indicate differences in empty procapsid clearance across the GSH chromatography step.

FIG. 12: A scalable and robust enterovirus purification process involving a clarification of cell culture harvest, an optional lysis step prior to harvest, the GSH affinity chromatography step, an optional anion exchange (AEX) polishing chromatography step, a solution adjustment, the cation exchange (CEX) chromatography step, a buffer exchange step using either tangential flow filtration (TFF) or size exclusion chromatography (SEC), and a final filtration step is described. The sample name for the product from each unit operation that is forwarded to the next step is shown.

FIG. 13: Reducing 12% acrylamide Bis-Tris SDS-PAGE with silver stain of purification process in FIG. 12 using Batch 4 as an example with GSH, AEX, solution adjustment, CEX, TFF, and filtration steps to produce purified virus. All samples loaded neat. VP0 detected in GSH elution, AEX FT, and CEX Feed samples, but is cleared in the CEX elution. The CEX strip contains mostly empty procapsids with high VP0 content. Final purified virus has high purity with only VP1, VP2, VP3 bands detected.

FIG. 14: Reducing 12% acrylamide Bis-Tris SDS-PAGE with silver stain of GSH elution, CEX elution, and Purified Virus samples from Batch 1-4. Sample loading normalized by volume concentration factor. The GSH step from batch 1-3 clears VP0, but some VP0 remains in Batch 4. As shown in FIG. 14, the CEX step clears empty procapsids and the final purified virus samples from Batch 1-4 all have similar viral protein distribution and high purity. Faint VP0 band detectable in UC pure sample.

FIG. 15: Comparison of the capillary electrophoresis quantitative western VP0/VP4 signal ratio detected with an anti-VP4 pAb for clarified harvest, GSH elution, and purified virus samples from Batches 1-4 relative to ultracentrifugation purified virus. Batch 1-3 empty procapsids were cleared across the GSH step while Batch 4 empty procapsids were cleared across CEX steps. The Batch 1-4 purified virus empty procapsid/full mature virus particle ratios were all ˜10× lower than the ultracentrifuge purified virus.

FIG. 16: Example RP-HPLC chromatogram for Batch 1 purified virus as detected using an acetonitrile gradient reverse-phase on an H-Class BIOshell C4 column (Waters). Viral proteins (VP1-4) are identified, as confirmed by mass spectrometry. Estimation of empty procapsid to full mature virion ratio by VP0:VP2 peak area ratio.

FIG. 17: Genome (RT-qPCR) and particle (HPSEC) to infectivity (plaque) ratios for Batches 1-4 and an ultracentrifuge purified virus (UC Pure). See Table 6 for analytical results.

FIG. 18: Example CE-SDS electropherogram showing the separation and relative migration times of the VP1, VP2, VP3 and VP4 in the Batch 4 purified virus sample.

### DETAILED DESCRIPTION OF THE INVENTION

Affinity chromatography is a purification method used to selectively bind and purify a target protein or biomolecule from a complex solution of impurities using an affinity ligand immobilized to a stationary phase. Glutathione (GSH) affinity chromatography was originally developed for the purification of recombinant Glutathione-S-transferase (GST)-tagged proteins due to the interaction of the immobilized glutathione ligand with a GST fusion protein [9], but not for untagged biomolecules such as enteroviruses, which do not contain GST or any similar protein sequences. Herein, we demonstrate the use of GSH affinity chromatography for the purification of full mature enterovirus (e.g., CVA21) virions.

Purified full mature CVA21 virus specifically binds to glutathione resin and can be eluted with free reduced glutathione through competitive displacement or a solution with high conductivity. Using a GSH affinity chromatography procedure, CVA21 was purified directly from clarified infected host-cell culture harvest in serum-containing media, with high infectivity yield (˜100%) and impurity clearance (>99.9% BSA and HCP reduction). Unexpectedly, it was discovered that mature CVA21 virions were enriched in the glutathione chromatography elution and that empty CVA21 procapsids flowed-through during the loading step. An additional cation exchange chromatography step, operated at bind and elute mode at lower pH, can also provide further clearance of empty procapsids and residual impurities.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The term “about”, when modifying the quantity (e.g., mM, or M), potency (genome/pfu, particle/pfu), purity (ng/ml), ratio of a substance or composition, the pH of a solution, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

As used herein, “x% (w/v)” is equivalent to x g/100 ml (for example, 5% w/v equals 50 mg/ml).

“CVA21” refers to Coxsackievirus A 21. One skilled in the art would understand that viruses may undergo mutation when cultured, passaged or propagated. The CVA21 may contain these mutations. Examples of CVA21 include but are not limited to the Kuykendall strain (GenBank accessions nos. AF546702 and AF465515), and Coe strain (Lennette et. al. Am J Hyg. 1958 Nov;68(3):272-87.) with or without mutations (e.g., SEQ ID NO: 1, or SEQ ID NO: 1 with position 7274 as C, and/or position 7370 as U) . The CVA21 may be a homogenous or heterogeneous population with none, or one or more of these mutations.

When referring to the genus or species of enteroviruses, one skilled in the art would understand that viruses may undergo mutation when cultured, passaged or propagated. The enterovirus may contain these mutations. Examples of the specific enteroviruses include but are not limited to the those listed in GenBank or UnitPro data bases with or without mutations. The enterovirus may be a homogenous or heterogeneous population with none, or one or more of these mutations.

“genome to infectivity ratio” refers to CVA21 RNA genome as measured by a genome RT-qPCR assay (genome/ml) divided by the infectivity (pfu/ml) as measured by a plaque assay. Viral plaque assays determine the number of plaque forming units (pfu) in a virus sample. One skilled in the art can develop various plaque assays to determine the infectivity (pfu/ml) of CVA21 after infection of a cell line. In one embodiment, the cell line is SK-MEL-28 cell line (ATTC deposit HTB-72). An example of a plaque assay is provided in example 6. The genome RT-qPCR assay measures the genome copies per ml using RT-qPCR methods with primers and probes targeting a CVA21 viral protein gene (e.g. example 6).

“particle to infectivity ratio” refers to CVA21 RNA genome as measured by HPSEC assay (particle/ml) divided by the infectivity (pfu/ml) as measured by a plaque assay. Viral plaque assays determine the number of plaque forming units (pfu) in a virus sample. One skilled in the art can develop various plaque assays to determine the infectivity (pfu/ml) of CVA21 after infection of a cell line. In one embodiment, the cell line is SK-MEL-28 cell line (ATTC deposit HTB-72). An example of a plaque assay is provided in example 6. The virus HPSEC assay determines the particle concentration (particle/ml) of CVA21. An example of this assay is provided in example 6.

“VP0 to VP2 ratio” refers to the ratio of the peak area of the CVA21 VP0 protein and VP2 protein as determined by reverse phase-HPLC or UPLC method. An example of the method is described in example 6.

“Stationary phase” is meant any surface to which one or more glutathione ligands can immobilize to. The stationary phase may be a suspension, purification column, a discontinuous phase of discrete particles, plate, sensor, chip, capsule, cartridge, resin, beads, monolith, gel, a membrane, or filter etc. Examples of materials for forming the stationary phase include mechanically stable matrices such as porous or non-porous beads, inorganic materials (e.g., porous silica, controlled pore glass (CPG) and hydroxyapatite), synthetic organic polymers (e.g., polyacrylamide, polymethylmethacrylate, polystyrene-divinylbenzene, poly(styrenedivinyl)benzene, polyacrylamide, ceramic particles and derivatives of any of the above) and polysaccharides (e.g., cellulose, agarose and dextran). See Jonsson, J. C.; Rydén, L. Protein Purification; Wiley: N.Y., 1998.

By “binding” an enterovirus to a stationary phase is meant exposing the enterovirus of interest to the stationary phase under appropriate conditions (pH and/or conductivity) such that the enterovirus is reversibly associated with the stationary phase by interactions between the enterovirus and glutathione immobilized on the stationary phase.

The term “equilibration solution” refers to a solution to equilibrate the stationary phase prior to loading the enterovirus on the stationary phase. The equilibration solution can comprise one or more of a salt and buffer, and optionally a surfactant. In one embodiment, the equilibration solution is the same condition as the loading solution comprising the enterovirus.

The term “loading solution” is the solution which is used to load the composition comprising the enterovirus of interest and one or more impurities onto the stationary phase. The loading solution may optionally further comprise one or more of a buffer, salt and surfactant.

The term “wash solution” when used herein refers to a solution used to wash or re-equilibrate the stationary phase, prior to eluting the enterovirus of interest. For washing, the conductivity and/or pH of the wash solution is/are such that the impurities (such as empty enterovirus pro-capsid, BSA, or HCP etc.) are removed from the stationary phase. For re-equilibration, the wash solution and elution solution may be the same, but this is not required. The wash solution can comprise one or more of a salt and buffer, and optionally a surfactant and/or reducing agent such as PS-80 and/or DTT.

The “elution solution” is the solution used to elute the enterovirus of interest from the stationary phase. The elution solution can comprise one or more of a salt, buffer and free reduced glutathione, optionally a surfactant and/or reducing agent such as DTT. The presence of one or more of free reduced glutathione (GSH), salt, buffer of the elution solution is/are such that the enterovirus of interest is eluted from the stationary phase.

A “strip solution” is a solution used to dissociate strongly bound components from the stationary phase prior to regenerating a column for re-use. The strip solution has a conductivity and/or pH as required to remove substantially all impurities and the enterovirus from the stationary phase. The strip solution can comprise one or more of a salt, buffer and GSH, and optionally a surfactant and/or reducing agent.

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mS/cm, and can be measured using a conductivity meter sold, e.g., within the GE Healthcare Akta System. The conductivity of a solution may be altered by changing the concentration of ions therein. For example, the concentration of a buffering agent and/or concentration of a salt (e.g. NaCl or KCl) in the solution may be altered in order to achieve the desired conductivity. Preferably, the salt concentration of the various buffers is modified to achieve the desired conductivity as in the Examples below.

By “purifying” an enterovirus of interest or “purified composition” is meant increasing the degree of purity of the enterovirus in the composition by removing (completely or partially) at least one impurity from the composition. The impurity can be empty procapsids, BSA, host cell components such as serum, proteins or nucleic acids, cellular debris, growth medium etc.. The term is not intended to refer to a complete absence of such biological molecules or to an absence of water, buffers, or salts or to components of a pharmaceutical formulation that includes the enterovirus.

As used herein, “glutathione is immobilized to a stationary phase” refers to a glutathione covalently attached to a stationary phase through conjugation of one or more reactive groups. In one embodiment, the glutathione stationary phase is a glutathione conjugated to the stationary phase through the thiol group of the glutathione.

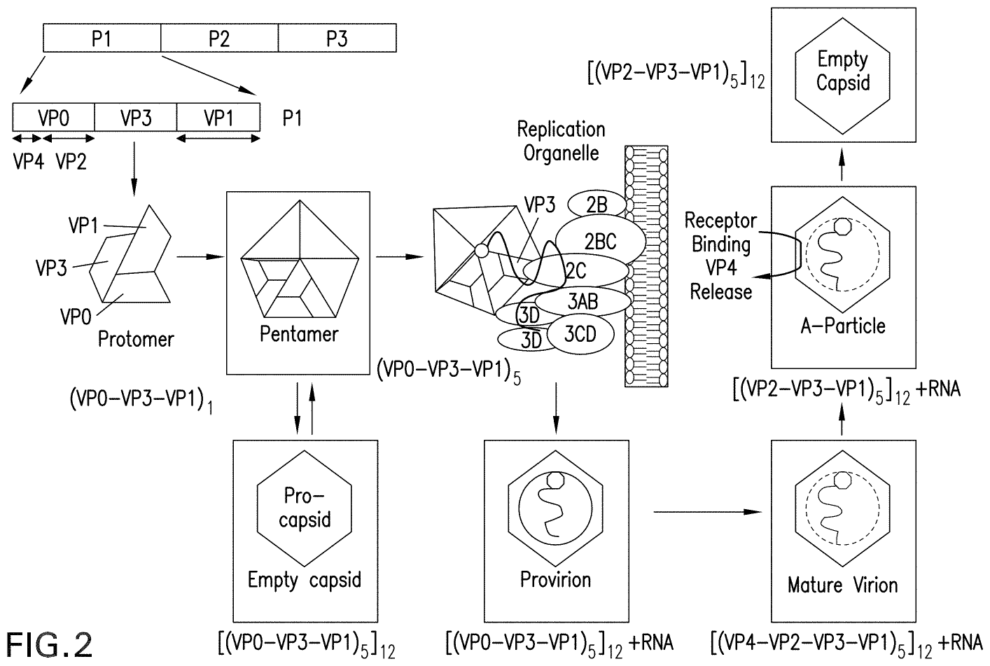

“Surfactant” is a surface active agent that is amphipathic in nature. “Mature virion” “full mature virion”, “full mature virus” or “full mature virus particle”, “full mature enterovirus”, “mature enterovirus”, “mature virus particle” refers to the mature enterovirus virion [(VP4-VP2-VP3-VP1)₅]₁₂+RNA as described in FIG. 2. Examples of the CVA21 VP1-VP4 sequence are described in Table 11.

“Empty capsid” refers to procapsid [(VP0-VP3-VP1)₅]₁₂, or degraded A-particle [(VP2-VP3-VP1)₅]₁₂ according to FIG. 2. An example of the VP0 sequence of CVA21 is in UnitPro Data Base accession no. P22055.

“Full capsid” refers to mature virion or provirion [(VP0-VP3-VP1)₅]₁₂+RNA as described in FIG. 2.

“Total VP1+VP2+VP3+VP4 peak area/total peak area” is the sum of the peak areas for VP1, VP2, VP3 and VP4 viral proteins of CVA21 divided by the total peak area (peak area of all quantifiable peaks above detection limit) in the Capillary Electrophoresis (CE)-SDS electropherogram. An Example of the CE-SDS method is in example 6.

“Impurity” refers to a material different from the desired enterovirus. The impurity can be a serum (i.e. BSA), Host Cell Protein (HCP), Host Cell DNA (HC-DNA), non-infectious virus-related particles including VP0-containing enterovirus (protomers, pentamers, provirions, procapsids), VP2-containing enterovirus (A-particles, or degraded A-particles). In one embodiment, the desired enterovirus is full mature enterovirus (e.g. full mature CVA21).

“TCID₅₀/ml” (Tissue Culture Infectious Dose 50%/mL) refers to the concentration of infectious organisms in the inoculum determined from the dilution at which the inoculum infects 50% of the target cultures. An example of a TCID₅₀ assay for CVA21 is provided in example 6.

“Treat” or “treating” cancer as used herein means to administer the pharmaceutical compositions of the invention to a subject having cancer, or diagnosed with cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)).

“per treatment” as used herein refers to administration of the pharmaceutical compositions of the invention to a patient on a single day. The pharmaceutical composition may be administered daily, intermittently (a few days a week, once a week, every two weeks, every three weeks, etc.)

### Compositions and Pharmaceutical Compositions of CVA21

The present invention also provides pharmaceutical compositions of the purified compositions of CVA21 and a pharmaceutically acceptable excipient. The CVA21 can be a mixture comprising one or more of a full mature CVA21 virion [(VP4-VP2-VP3-VP1)₅]₁₂+RNA, empty procapsid [(VP0-VP3-VP1)₅]₁₂, degraded A-particle [(VP2-VP3-VP1)₅]₁₂, A-particle [(VP2-VP3-VP1)₅]₁₂+RNA, provirion [(VP0-VP3-VP1)₅]₁₂+RNA, protomer (VP0-VP3-VP1)₁, and pentamer (VP0-VP3-VP1)₅. In one embodiment, the CVA21 comprises full mature CVA21 virion [(VP4-VP2-VP3-VP1)₅]₁₂+RNA. In one embodiment, the CVA21 comprises full mature CVA21 virion [(VP4-VP2-VP3-VP1)₅]₁₂+RNA and empty procapsid [(VP0-VP3-VP1)₅]₁₂.

In one embodiment, the genome to infectivity ratio of the composition is less than about 5000 genome/pfu. In one embodiment, the genome to infectivity ratio of the composition is less than about 4000 genome/pfu. In one embodiment, the genome to infectivity ratio of the composition is less than about 3000 genome/pfu. In one embodiment, the genome to infectivity ratio of the composition is less than about 2000 genome/pfu. In one embodiment, the genome to infectivity ratio of the composition is less than about 1000 genome/pfu. In one embodiment, the genome to infectivity ratio is about 200-2000 genome/pfu. In one embodiment, the genome to infectivity ratio of the composition is less than about 800 genome/pfu. In one embodiment, the genome to infectivity ratio is about 400-700 genome/pfu. In another embodiment, the genome to infectivity ratio is about 400-800 genome/pfu. The present invention also provides a composition of CVA21, wherein the particle to infectivity ratio is less than about 5000 particle/pfu. In one embodiment, the particle to infectivity ratio is less than about 4000 particle/pfu. In one embodiment, the particle to infectivity ratio is less than about 3000 particle/pfu. In one embodiment, the particle to infectivity ratio is less than about 2000 particle/pfu. In one embodiment, the particle to infectivity ratio is less than about 1000 particle/pfu. In one embodiment, the particle to infectivity ratio is less than about 900 particle/pfu. In one embodiment, the particle to infectivity ratio is less than about 800 particle/pfu. In one embodiment, the particle to infectivity ratio is less than about 700 particle/pfu. In one embodiment, the particle to infectivity ratio is less than about 600 particle/pfu. In one embodiment, the particle to infectivity ratio is less than about 500 particle/pfu. In one embodiment, the particle to infectivity ratio is about 200-600 particle/pfu. In one embodiment, the particle to infectivity ratio is about 200-500 particle/pfu. In one embodiment, the particle to infectivity ratio is about 200-800 particle/pfu.

The present invention provides pharmaceutical compositions of purified CVA21, wherein the VP0 to VP2 ratio is less than about 0.01. In one embodiment, the VP0 to VP2 ratio is about 0.0005-0.01. In one embodiment, the VP0 to VP2 ratio is about 0.0005-0.005. In another embodiment, the VP0 to VP2 ratio is about 0.001-0.003. In another embodiment, the total VP1+VP2+VP3+VP4 peak area/total peak area is at least 95%. In one embodiment, the host cell DNA is less than about 10 ng/ml. In another embodiment, the host cell DNA is less than about 0.5 ng/ml. In another embodiment, the amount of host cell DNA in the composition is less than about 10,000 pg/dose, with about 5E7 pfu CVA21 per dose. In another embodiment, the amount of host cell DNA in the composition is about 0.05-10,000 pg/dose, with about 5E7 pfu CVA21 per dose. In another embodiment, the amount of host cell DNA in the composition is about 0.05-10 pg/dose, with about 5E7 pfu CVA21 per dose. In another embodiment, the amount of host cell DNA in the composition is about 0.05-1 pg/dose, with about 5E7 pfu CVA21 per dose. In another embodiment, the amount of host cell DNA in the composition is about 100-10,000 pg/dose, with about 5E7 pfu CVA21 per dose. In another embodiment, the amount of bovine serum albumin in the composition is less than about 10 ng/ml. In one embodiment, the bovine serum albumin is less than about 50,000 pg/dose, with about 5E7 pfu CVA21 per dose. In another embodiment, the bovine serum albumin is about 500-50,000 pg/dose, with about 5E7 pfu CVA21 per dose. In another embodiment, the bovine serum albumin is about 50-100 or 50-150 pg/dose, with about 5E7 pfu CVA21 per dose. In another embodiment, the bovine serum albumin is less than about 150 pg/dose, with about 5E7 pfu CVA21 per dose.

Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbitol, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

In one aspect, the pharmaceutical composition is for intratumoral administration In another embodiment, the pharmaceutical composition is for intravesical administration. In one embodiment, the dose per treatment is up to about 3E8 TCID₅₀ or about 5E7 pfu depending on the size, location and number of the tumors. In another embodiment, the dose per treatment is about 3E7 to 3E8 TCID₅₀ or about 5E6 to 5E7 pfu. Injection techniques which increase or maximize the distribution of the virus throughout the tumor may offer improved therapeutic outcomes. For example, in the treatment of melanoma and other solid tumors, multiple lesions may be injected in a dose hyper-fraction pattern, starting with the largest lesion(s) (2.0 mL injected into tumors>2.5 cm, 1.0 mL into 1.5 to 2.5 cm; 0.5 mL into 0.5 to 1.5 cm) to a 4.0 mL maximum. Following initial injection with CVA21, any injected lesion that reduces in diameter to <0.5 cm may be injected with 0.1 mL of CVA21 as per the treatment schedule until the lesion completely resolves. In another embodiment, the pharmaceutical composition is for intravenous administration. In one embodiment, the dose per treatment is about 1E9 TCID₅₀ or about 1.5E8 pfu.

In another aspect, the pharmaceutical composition has a potency of about 1E5 to 1E12 TCID₅₀/ml or pfu/ml. In one embodiment, the pharmaceutical composition has a potency of about 1E6 to 1E12 TCID₅₀/ml or pfu/ml. In one embodiment, the pharmaceutical composition has a potency of about 1E7 to 1E11 TCID₅₀/ml or pfu/ml. In one embodiment, the pharmaceutical composition has a potency of about 1E7 to 8E7 TCID₅₀/ml. In one embodiment, the pharmaceutical composition has a potency of about 5E7 to 8E7 TCID₅₀/ml. In one embodiment, the pharmaceutical composition has a potency of about 7.5E7 TCID₅₀/ml. The CVA21 virus TCID50 assay is disclosed in WO 2015/127501, and in Example 6. In another aspect, the pharmaceutical composition has a potency of 5E6 to 5E7 pfu/ml. In another aspect, the pharmaceutical composition has a potency concentration of 1E7 to 3E7 pfu/ml. In another aspect, the pharmaceutical composition has a potency concentration of 1.1E7 pfu/ml. The potency can be measured by the plaque assay in Example 6.

### Methods of Treating Cancer

In a further aspect, the invention provides a method for treating cancer in a patient comprising administering the pharmaceutical composition of the invention to the patient. In one embodiment, the pharmaceutical composition is administered intratumorally. In another embodiment, the pharmaceutical composition is administered intravesically. In one embodiment, the dose per treatment is up to about 3E8 TCID₅₀ or about 5E7 pfu. In one embodiment, the dose per treatment is up to about 3E7 TCID₅₀ or about 5E6 pfu. In one embodiment, the dose per treatment is up to about 1E8 TCID₅₀ or about 1.5E7 pfu. In another embodiment, the pharmaceutical composition is administered intravenously. In one embodiment, the dose per treatment is 1E9 TCID₅₀ or about 1.5E8 pfu. In a further embodiment, the pharmaceutical composition is administered on intermittent days.

Cancers that may be treated by the pharmaceutical compositions of the invention include, but are not limited to: Cardiac cancers: sarcoma (angiosarcoma, fibrosarcoma, rhabdomyosarcoma, liposarcoma), myxoma, rhabdomyoma, fibroma, lipoma and teratoma; Lung cancers: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancers: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract cancers: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver cancers: hepatoma (hepatocellular carcinoma), cholangiocarcinoma, hepatoblastoma, angiosarcoma, hepatocellular adenoma, hemangioma; Bone cancers: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system cancers: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancers: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic cancers: blood (myeloid leukemia (acute and chronic), acute lymphoblastic leukemia, chronic lymphocytic leukemia, myeloproliferative diseases, multiple myeloma, myelodysplastic syndrome); hematopoietic tumors of the lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoetic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma. In one embodiment, the forgoing cancers are advanced, unresectable or metastatic.

In one embodiment, cancers that may be treated by pharmaceutical compositions of the invention include, but are not limited to: non-small cell lung cancer, bladder carcinoma, melanoma, triple negative breast cancer, hepatocellular carcinoma, gastric carcinoma, head and neck squamous cell carcinoma, and cutaneous squamous cell carcinoma.

### Glutathione Affinity Chromatography

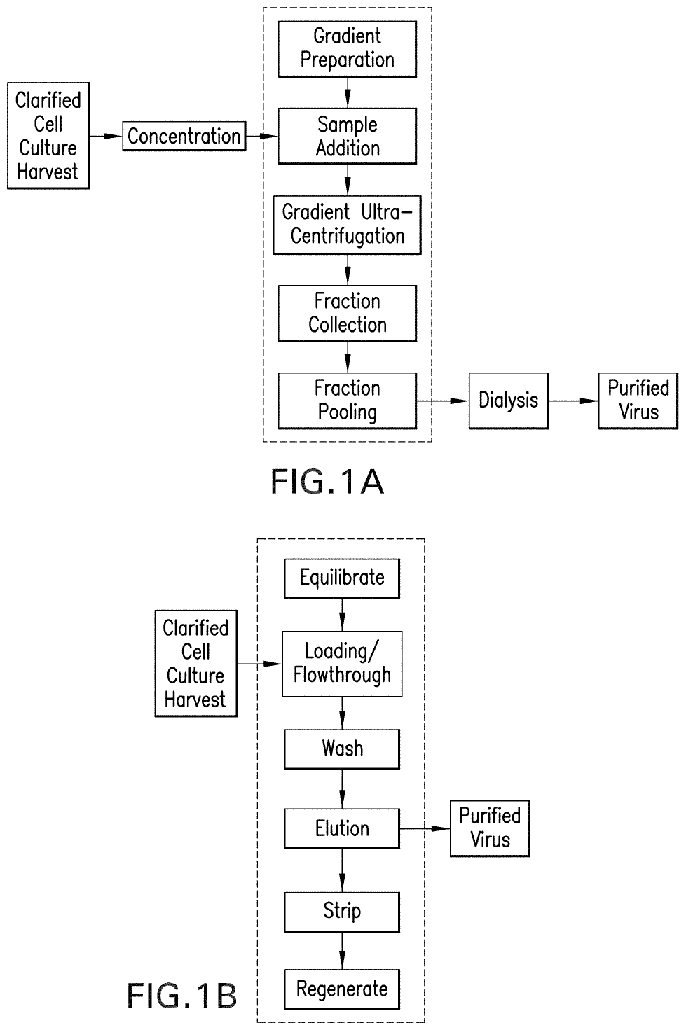

The invention provides a scalable and robust method for purifying enteroviruses using glutathione affinity chromatography (FIG. 1B). In one embodiment, the glutathione affinity chromatography stationary phase comprises a glutathione (GSH) immobilized to the surface of a stationary phase. Glutathione (also named L-glutathione, reduced glutathione, or GSH) is a biologically-active tri-peptide (glutamic acid-cysteine-glycine) in human cells used to control redox potential and is involved in many cellular functions [8]. GSH has the following chemical structure and name:

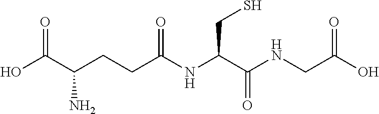 [SMILES: N[C@@H](CCC(=O)N[C@@H](CS)C(=O)NCC(=O)O)C(=O)O]

(2S)-2-amino-5-[[(2R)-1-(carboxymethylamino)-1-oxo-3-sulfanylpropan-2-yl]amino]-5-oxopentanoic acid.

The glutathione can be immobilized to the stationary phase through conjugation of the SH group using maleimide, haloacetyl, pyridyl disulfide, epoxy or other similar sulfhydryl-reactive based chemistries. See Stenzel M H, ACSMacro Letters, 2, 14-18 (2013). GSH resin is also commercially available through several vendors (Cytiva, Thermo, Qiagen, Sigma).

In batch mode, the stationary phase is utilized free in solution. For utilization in flow mode, the stationary phase is packaged into a column, capsule, cartridge, filter or other support and a flowrate of about 1-500 cm/hr is used.

In one aspect, the invention provides a method of purifying an enterovirus comprising the steps of:
a. binding an enterovirus to a stationary phase using a loading solution, wherein glutathione is immobilized to the stationary phase;
b. eluting the enterovirus from the stationary phase with an elution solution.

In one embodiment, prior to step (a), equilibrating the stationary phase with an equilibration solution is performed. In one embodiment, one or more impurities are in the flowthrough of step a).

In another aspect of the method, after step a) but prior to step (b), it further comprises step i) of washing the stationary phase with one or more wash solutions. In one embodiment, one or more impurities are removed from the wash step. In another embodiment, step (i) comprises a first wash step with a wash solution having a conductivity higher than the equilibration solution or loading solution. In another embodiment, step (i) comprises a second wash step with a wash solution having a conductivity lower than the wash solution in the first wash step. In a further embodiment, the conductivity of the elution solution is the same as the wash solution in the second wash step.

In one embodiment, the loading solution, equilibration solution, wash solution or elution solution comprises a salt, preferably a monovalent metal ion salt, such as NaCl or KCl. In another embodiment, the loading solution or equilibration solution comprises about 50-200 mM NaCl or KCl. In a another embodiment, the loading solution or equilibration solution comprises about 100 mM NaCl or KCl.

In one embodiment, the wash solution comprises about 50-400 mM NaCl or KCl. In another embodiment, the wash solution comprises about 350-450 mM NaCl or KCl. In another embodiment, the wash solution comprises about 400-500 mM NaCl or KCl. In a further embodiment, the wash solution comprises about 400 mM NaCl or KCl. In a further embodiment, a first wash solution comprises about 100-500 mM NaCl or KCl and a second wash solution comprises about 50-500 mM NaCl or KCl. In a further embodiment, a first wash solution comprises about 350-500 mM NaCl or KCl and the second wash solution comprises about 50-150 mM NaCl or KCl. In a further embodiment, the first wash solution comprises about 400 mM NaCl or KCl and the second wash solution comprises about 75 mM NaCl or KCl. In a further embodiment, the second wash solution comprises about 50-150 mM NaCl or KCl. In a further embodiment, the second wash solution comprises about 100 mM NaCl or KCl.

The elution step may be performed with a solution with high ionic strength or high conductivity, low pH (for example pH about 5-7), or in the presence of free GSH, or a combination thereof. In one embodiment, the elution solution comprises about 0.5-1 M of monovalent salt such as NaCl or KCl. In one embodiment, the elution solution comprises about 0.5 M of NaCl or KCl. In one embodiment, the elution solution comprises about 50-500 mM of NaCl or KCl. In another embodiment, the elution solution comprises about 0.1-100 mM glutathione. In another embodiment, the elution solution comprises about 0.1-50 mM glutathione. In another embodiment, the elution solution comprises about 0.1-25 mM glutathione. In another embodiment, the glutathione in the elution solution is about 1 mM. In one embodiment, the elution solution comprises about 0.5-5 mM glutathione and about 75-150 mM NaCl or KCl. In one embodiment, the elution solution comprises about 0.5-25 mM glutathione and about 50-500 mM NaCl or KCl. In another embodiment, the elution solution comprises about 0.1-100 mM glutathione and about 75-150 mM NaCl, and optionally about 0.001-1% w/v PS-80. In yet a further embodiment, the elution solution comprises about 100 mM NaCl, about 1 mM glutathione, and about 0.005% w/v PS80.

In one embodiment, one or more of the loading solution, equilibration solution, wash solutions and elution solution has a pH of about 6.5-8.5. In a another embodiment, one or more of the loading solution, equilibration solution, wash solutions and elution solution has a pH of about 7-8. In a another embodiment, one or more of the loading solution, equilibration solution, wash solutions and elution solution has a pH of about 8. In a further embodiment, one or more of the loading solution, equilibration solution, wash solutions and elution solution has a pH of about 6-9. In yet a further embodiment, one or more of the loading solution, equilibration solution, wash solutions and elution solution has a pH of about 5-10.

In one embodiment, one or more of the loading solution, equilibration solution, wash solutions and elution solution further comprises a surfactant. In another embodiment, the surfactant is PS-80 or PS-20. In another embodiment, the surfactant is about 0.001-1% w/v PS-80. In another embodiment, the surfactant is about 0.001-0.1% w/v PS-80. In another embodiment, the surfactant is about 0.005% w/v PS-80. In one embodiment, one or more of the loading solution, wash solutions and elution solution further comprises EDTA, or a reducing agent such as DTT or ß-mercaptoethanol. In another embodiment, the reducing agent is DTT. In another embodiment, the DTT is at about 0.1-10 mM. In another embodiment, the DTT is at about 0.1-5 mM. In another embodiment, the DTT is at about 1 mM.

In one embodiment, the desired enterovirus is full mature enterovirus. In one embodiment, the desired enterovirus is full mature CVA21. In one embodiment, at least the full mature enterovirus binds to the stationary phase upon loading the solution. In one embodiment, the purification process removes one or more impurities such as serum (i.e. BSA), HCP, HC-DNA, non-infectious virus-related particles including but not limited to VP0-containing enterovirus (protomers, pentamers, provirions, procapsids), VP2-containing enterovirus (A-particles, or empty capsids from degraded A-particles) through the flow-through or wash step. In a further embodiment, the purification process removes enterovirus procapsids (e.g., CVA21 procapsids). In another embodiment, the purification process results in a composition comprising high purity (>99% pure) full mature enterovirus (e.g. full mature CVA21 virions).

The methods of the invention can be used in conjunction with other chromatography or purification steps to remove impurities. In one embodiment, the purification process removes one or more cell culture impurities such as serum (i.e. BSA), HCP, or HC-DNA, through the flow-through or wash step. In another embodiment, the purification process removes one or more impurities such as protomers, pentamers, provirions, procapsids, A-particles, and degraded A-particles through the flow-through or wash step. In another aspect, the invention provides a purified composition of the enterovirus obtainable by or produced by the foregoing purification steps and/or embodiments of the invention.

In one embodiment, the method of the invention is comprised of the following steps: a) loading a cell culture media comprising enterovirus to a stationary phase, preferably at a loading of 1-1000 L-harvest medium/L-stationary phase, wherein cell culture media impurities and/or empty procapsids are purified through the flowthrough b) washing the stationary phase with a wash solution to remove residual impurities, c) eluting the enterovirus from the stationary phase with an elution solution comprising reduced glutathione, preferably about 0.1-50 mM, a buffer volume of preferably 2-20 times the column or membrane volumes. The method may optionally comprise additional steps d) to strip the stationary phase with a strip solution that removes strongly bound impurities from the stationary phase, and e) regenerating the stationary phase. In one embodiment, the strip solution comprises about 1-50, or 5-50 mM GSH. In another embodiment, the strip solution comprises about 10 mM GSH. In one embodiment, the strip solution comprises about 500-1500, or 1000-2000 mM NaCl.

### Enterovirus

Any suitable source of enterovirus may be used in the methods of the invention [1]. The enterovirus particle can be poliovirus, Group A Coxsackievirus, Group B Coxsackievirus, echovirus, rhinovirus, and numbered enterovirus. In one embodiment, the enterovirus is a Group A, B or C enterovirus. In one embodiment, the enterovirus is a Group C enterovirus. In one embodiment, the enterovirus is a Group A or B Coxsackievirus. In another embodiment, the enterovirus is Group A Coxsackievirus. In one embodiment, the Group C enterovirus is a Group A Coxsackievirus selected from the group consisting of CVA1, CVA11, CVA13, CVA15, CVA17, CVA18, CVA19, CVA20a, CVA20b, CVA20c, CVA21, CVA22 and CVA24. In one embodiment, the Group A Coxsackievirus is selected from the group consisting of CVA13, CVA15, CVA18, CVA20, and CVA21. Various suitable strains of these viruses may be obtained from the American Type Culture Collection (ATCC), 10801 University Blvd., Manassas, Va. 20110-2209 USA, such as material deposited under the Budapest Treaty on the dates provided below, and is available according to the terms of the Budapest Treaty: Coxsackie group A virus, strain CVA13, ATCC No.: PTA-8854, deposited Dec. 10, 2007; Coxsackie group A virus, strain CVA15 (G9), ATCC No.: PTA-8616, deposited Aug. 15, 2007; Coxsackie group A virus, strain CVA18, ATCC No.: PTA-8853, deposited Dec. 20, 2007; and Coxsackie group A virus, strain CVA21 (Kuykendall), ATCC No.: PTA-8852, deposited Dec. 20, 2007. Other Group A Coxsackie virus under Group C enterovirus referenced in the literature include but are not limited to CVA1 (GenBank accession no. AF499635, Dalldorf et al., 1949), CVA11 (GenBank accession no. AF499636), CVA17 (GenBank accession no. AF499639), CVA19 (GenBank accession no. AF499641) , CVA20 (GenBank accession no. AF499642), CVA20a (Sickles et al., 1959), CVA20b (Sickles et al., 1959), CVA20c (Abraham and Cheever, 1963), CVA22 (Sickles et al., 1959; (GenBank accession no. AF499643), and CVA24 (Mirkovic et al., 1974; (GenBank accession no. EF026081). In a preferred embodiment, the enterovirus is a Coxsackievirus A21.

In another embodiment, the enterovirus is a Group B enterovirus. In another embodiment, the Group B enterovirus is echovirus. In another embodiment, the Group B enterovirus is echovirus-1 (EV-1). Examples of echovirus-1 include those with GenBank accession nos. AF029859, AF029859.2 and AF250874.

In another embodiment, the enterovirus is a Group B Coxsackievirus. In a further embodiment, the Group B Coxsackievirus is Coxsackievirus B3 (CVB3) or Coxsackievirus B4 (CVB4). In a further embodiment, the enterovirus is a Rhinovirus A, B or C. In another embodiment, the enterovirus is Rhinovirus A or B. In yet a further embodiment, the enterovirus is Human Rhinovirus 14 (HRV14). In yet a further embodiment, the enterovirus is Human Rhinovirus 1B or 35. An example of Human Rhinovirus 1B is Genbank accession no. D00239.1. An example of human Rhinovirus 35 is Genbank accession no. EU870473. In yet another embodiment, the enterovirus is Echovirus 1, Rhinovirus 1B, Rhinovirus 35, Coxsackievirus A 13 (CVA13), Coxsackievirus A 15 (CVA15), Coxsackievirus A 18 (CVA18), Coxsackievirus A 20b (CVA20b), or Coxsackievirus A 21 (CVA21). A summary of the current understanding of enterovirus morphogenesis and the role of glutathione in capsid assembly is detailed in FIG. 2. Furthermore, genetically modified enterovirus with transgene insertion, and inactivated enteroviruses can be used in the methods of the invention.

### EXAMPLES

The examples are presented in order to more fully illustrate the various embodiments of the invention. These examples should in no way be construed as limiting the scope of the invention recited in the appended claims.

### Example 1

### Purification of Coxsackievirus A21 with GSH Affinity Chromatography

The described GSH affinity chromatography procedure [FIG. 1B] was performed using an Akta Pure 150M FPLC system (Cytiva) with UNICORN system control software (Cytiva). A Cytiva 20 mL HiPrep column packed with GSH Sepharose 4 Fast Flow resin was used for purification of CVA21. The 20 mL GSH column was equilibrated with 5 column volumes (CV) of equilibration solution containing 15 mM Tris, 150 mM NaCl, 0.005% w/v PS80, pH 7.5 at a flow rate of 150 cm/hr and a 4 min residence time. The CVA21 clarified cell culture harvest was loaded to the column at a flow rate of 100 cm/hr and 6 min residence time until a column loading of 200-CVs was reached. The GSH column was washed at a flow rate of 150 cm/hr with 5-CVs of wash 1 buffer containing 15 mM Tris, 400 mM NaCl, 0.005% w/v PS-80, pH 8.0 and then 5-CVs of wash 2 buffer containing 15 mM Tris, 100 mM NaCl, 0.005% w/v PS-80, 1 mM DTT, pH 8.0. The bound CVA21 particles were eluted with 3-CVs of elution solution containing 15 mM Tris, 100 mM NaCl, 0.005% w/v PS-80, 1 mM DTT, 1 mM GSH, pH 8.0 through competitive displacement from the immobilized glutathione ligand with free GSH in the mobile phase at a flow rate of 150 cm/hr. The GSH column was stripped with 3-CVs of a buffer containing 15 mM Tris, 1000 mM NaCl, 0.005% w/v PS-80, 1 mM DTT, 10 mM GSH, pH 8.0 and regenerated with a 0.1 N NaOH, 1 M NaCl solution at a flow rate of 150 cm/hr. The resin may be reused to load additional harvest after re-equilibrating the column.

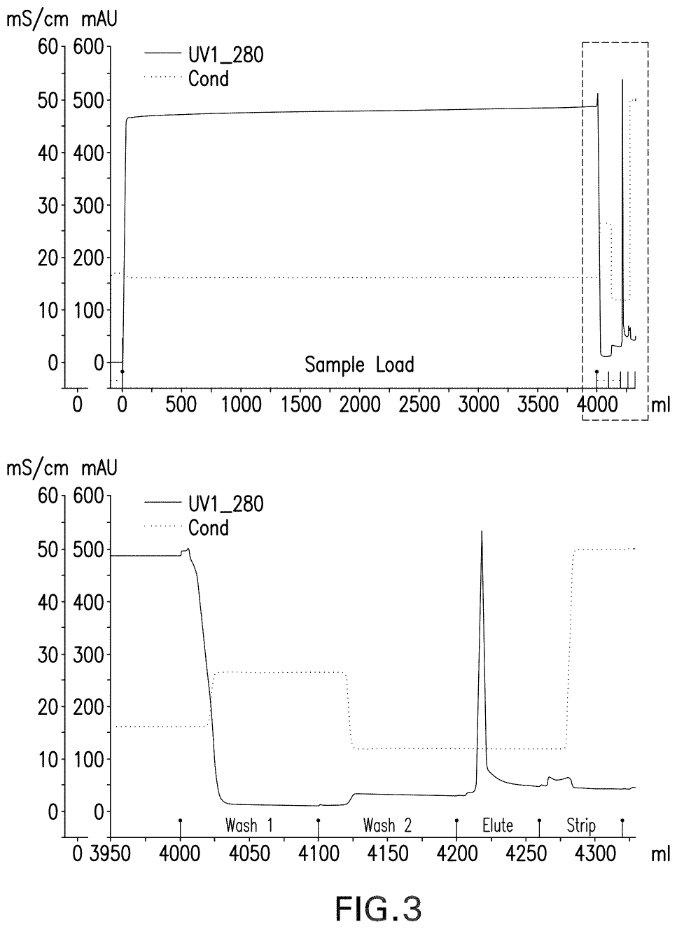

The chromatogram analyzed in UNICORN software depicts the absorbance trace at 280 nm in mAU (A280) and conductivity trace in mS/cm [FIG. 3]. The A280 signal did not change during the loading (GSH FT) of the clarified cell culture harvest, indicating consistent flow-through of impurities and undetectable breakthrough of virus particles over time. The column was washed with 5-CVs of a 400 mM NaCl containing wash buffer (GSH W1) to remove weakly or non-specifically bound impurities until the A280 reached baseline. Upon conditioning the column with wash 2 buffer (GSH W2), the CVA21 particles were eluted with 3-CVs of a buffer containing 1 mM free reduced GSH (GSH Elute), and a corresponding A280 peak was observed. The overall volume concentration factor of the GSH elution from the clarified harvest was 67-fold. The column was stripped with a strip solution containing 10 mM GSH and 1 M NaCl (GSH Strip) and a small peak was observed.

The clarified harvest and GSH chromatography elution samples were analyzed by a median tissue culture infectious dose (TCID50) assay for viral infectivity, a reverse-transcription quantitative polymerase chain reaction (RT-qPCR) assay for total viral genomes, and a capillary electrophoresis quantitative western blot (Protein Simple) using an anti-VP1 antibody for total particles. The GSH chromatography elution yields were calculated by the virus quantity in the GSH elution product divided by the quantity in the loaded cell culture harvest. The infectivity and viral genome yields were about 100% while the total particle yield was near 50% [Table 1]. This suggests that full, mature, infectious CVA21 virions bound to the immobilized GSH ligand and eluted from the column with excellent recovery, while a proportion of non-infectious viral particles were cleared.

TABLE 1

| | GSH Elution Yield |
|---|---|
| Virus Attribute | from Clarified Harvest |
| Viral Infectivity (TCID₅₀) | 106% |
| Viral Genomes (RT-qPCR) | 93% |
| Total Viral Particles | 49% |
| (anti-VP1 Western) | |

Enterovirus infected cells typically produce both infectious mature virions and non-infectious virus-related particles including disassembled protomers or pentamers, provirions, empty procapsids, A-particles, or degraded A-particles [FIG. 2]. A typical infected cell culture harvest may also contain host-cell related impurities, including host-cell proteins and host-cell DNA, and culture media-related impurities, including bovine serum albumen (BSA). Sodium dodecyl sulfate polyacrylamide electrophoresis (SDS-PAGE) was used to detect the viral and impurity protein bands in the GSH affinity chromatography fractions. The samples were prepared neat and mixed with 4× loading dye (BioRad) and 10× reducing agent (BioRad) before heating at 70° C. for 10 min to denature the samples. 20 μL of the denatured samples were added to a 10-well 12% acrylamide Bis-Tris NuPAGE gel (Thermo) and run at 200V for 60 min using an Invitrogen gel box and power supply system (Thermo). The gel was stained using a Pierce silver stain kit (Thermo) and imaged using a gel imager (BioRad).

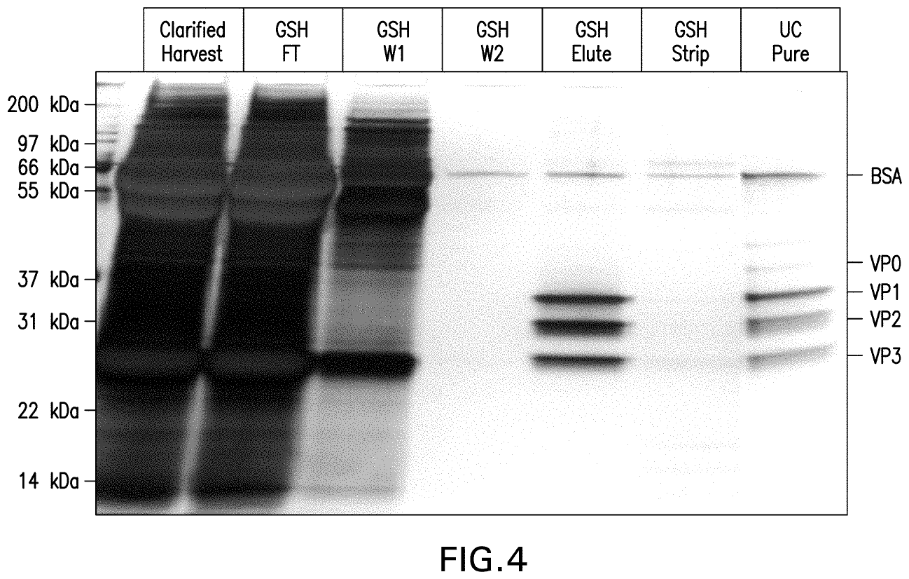

Analysis of SDS-PAGE showed that the clarified harvest contained a high concentration of bovine serum and host-cell protein impurities which mostly flowed-through during the sample load (GSH FT) [FIG. 4]. Additional impurities were removed during the first wash (GSH W1), and by the second wash, only a faint band of BSA (66.5 kDa) was detected (GSH W2). The following CVA21 viral capsid protein bands were visible in the GSH elution, as determined by their expected molecular weight (UniProt A0A0N9H7H0): VP1 (33.2 kDa), VP2 (29.9 kDa), and VP3 (26.6 kDa). The VP4 (7.5 kDa) band ran off the bottom of the gel due to its small molecular weight. In addition, very faint VP0 (37.3 kDa) was detectable by SDS-PAGE, signifying that the GSH elution contained virus particles that underwent VP0 cleavage into VP2 and VP4 after encapsidation of the genome. Thus, SDS-PAGE demonstrated that the GSH affinity chromatography selectively binds mature CVA21 virions that do not contain VP0 with efficient impurity clearance.

The GSH affinity chromatography purified virus was compared to virus purified by the conventional ultracentrifugation (UC) process using a cesium chloride (CsCl) gradient [FIG. 1A]. Before the UC step, the clarified cell culture harvest was concentrated 100-fold by tangential-flow filtration (TFF) using a 300 kDa PES hollow fiber filter (Repligen). The gradient was prepared in an ultracentrifuge tube using various density solutions of CsCl and the concentrated cell culture lysate was added to the top of the gradient. The samples were centrifuged at 67,000 g for 5 hrs and selected fractions were pooled and dialyzed across a 10 kDa membrane into a stabilizing buffer solution. Through SDS-PAGE analysis, VP0 and other unknown protein bands were detectable in the UC purified virus lane [FIG. 4], indicating the presence of empty procapsids and other impurities in the sample.

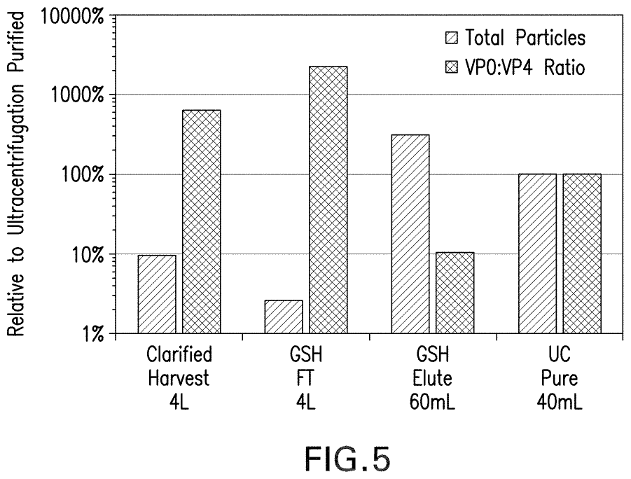

The GSH affinity chromatography and UC purified samples were analyzed by a capillary electrophoresis quantitative western blot (Protein Simple) using 2 primary antibodies: An anti-VP1 polyclonal antibody that binds to VP1 and an anti-VP4 polyclonal antibody that binds to both VP4 and VP0 proteins. VP1 is present in all virus particles containing protomer/pentamer subunits and the anti-VP1 peak area was used to estimate total virus particles. VP0 is only present in empty procapsids, provirions, and protomer/pentamer subunits (VP0-containing enterovirus particles), while particles with VP4 result from a VP0 cleavage to VP2+VP4 after encapsidation of the genome to form full, mature virions [FIG. 2]. Therefore, the ratio of the VP0:VP4 peak areas was used to estimate the VP0-containing enterovirus particle:full mature virion ratio. The total viral particles and VP0-containing particle/full mature virion ratio of the clarified harvest and GSH chromatography FT and elution were analyzed relative to the UC pure sample [FIG. 5]. In the clarified harvest, there were ˜10-fold fewer total particles with a ˜6-fold higher relative ratio of VP0-containing particle/full mature virion than the UC pure sample. During the GSH affinity chromatography loading step, viral particles were present in the GSH FT, but these particles had a high VP0-containing particle content. The total particles in the GSH elution increased by ˜30-fold from the clarified harvest due to the volume reduction, and the VP0:VP4 ratio was decreased by a factor of ˜60×. Compared to the UC purified virus, the GSH elution had a 10-fold lower VP0-containing enterovirus particle:full mature virion ratio. This demonstrated that GSH affinity chromatography selectively bound full mature virion particles containing VP4, while flowing though particles with VP0, and cleared empty procapsids more efficiently than the gradient ultracentrifugation method.

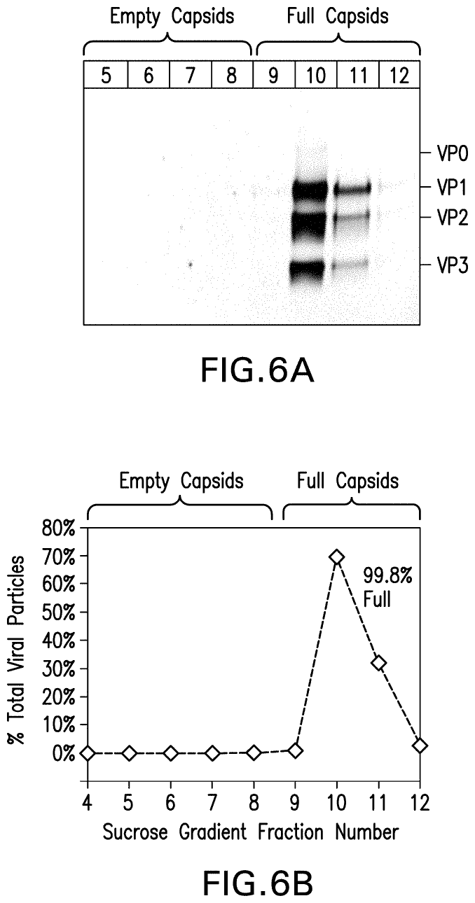

Sucrose gradient ultracentrifugation was run to confirm the presence of only full mature virion particles in the purified GSH elution sample. 1 mL of the GSH elution sample was loaded on top of a 15-42% w/v continuous sucrose gradient and centrifuged at 230,000 g for 100 min. 12×1 mL fractions were taken and analyzed by SDS-PAGE with silver stain and capillary electrophoresis quantitative western using an anti-VP1 primary antibody. Fractions 5-8 were expected to contain empty capsids, while fractions 9-12 were expected to contain full capsids. As shown in the SDS-PAGE gel, the viral proteins VP1, VP2, VP3 and no VP0 were detected in fractions 10-11, while no bands were detected in lanes 5-8 [FIG. 6A]. This indicated the GSH elution contained only full mature virions and no provirions or empty capsids. The distribution of total virus particles across the fractions measured by VP1 demonstrated that the GSH elution sample contained more than 99% full mature virions [FIG. 6B].

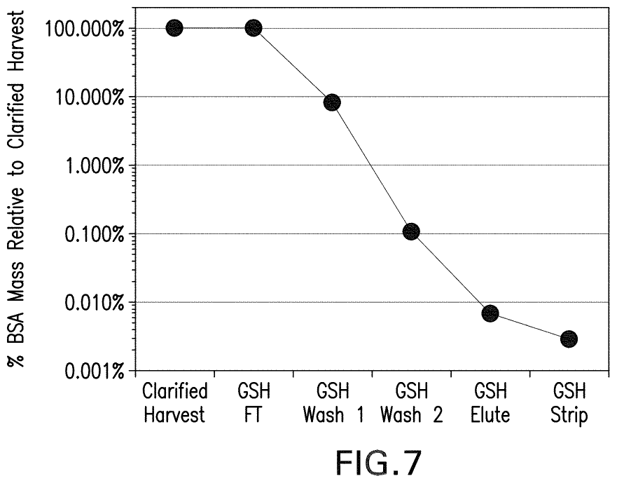

In addition to removing empty procapsids and other viral impurities, GSH affinity chromatography efficiently cleared serum and host cell impurities. BSA is a major component of the bovine serum used in the cell culture media. In typical cell culture media with serum, the BSA concentration is about 0.5-1.0 mg/mL and a significant reduction is required to meet a typical target of <50 ng BSA per dose. The GSH chromatography fractions were analyzed by capillary electrophoresis quantitative western blot (Protein Simple) and detected with an anti-BSA primary antibody (Bethyl) [FIG. 7] using a published procedure [16] with a primary incubation time of 90 min and secondary antibody incubation time of 60 min. Greater than 90% of the initial BSA mass flowed through during the GSH loading step, and weakly bound BSA was removed in the 2 wash steps. The percent reduction of GSH elution residual BSA from the clarified harvest was greater than 99.99%. The GSH elution HCP clearance, measured by capillary electrophoresis western blot with an anti-MRC-5 primary antibody (Cygnus), and HC-DNA clearance, measured by qPCR (See Analytical Example 6), were also evaluated [Table 2]. The percent reduction from clarified harvest was more than 99.9% for HCP and more than 99.8% for HC-DNA. These results establish that GSH affinity chromatography can be utilized for the efficient concentration and purification of mature, infectious CVA21 virions, while clearing non-infectious empty procapsids, other virus impurities, and cell culture related impurities.

TABLE 2

| | GSH Elution % Reduction |
|---|---|
| Impurity | from Clarified Harvest |
| Bovine Serum Albumen | 99.993% |
| Host Cell Protein | 99.986% |
| Host Cell DNA | 99.818% |

### Example 2

### Amplification and Purification of Enteroviruses with GSH Affinity Chromatography

Enteroviruses are a genus within the Picornavirus family consisting of small, positive sense, single stranded RNA viruses that share similar genomic and structural viral properties. To demonstrate the GSH affinity purification of enteroviruses, 8 different serotypes encompassing several enterovirus species including Enterovirus B, Enterovirus C, Rhinovirus A, and Rhinovirus B were evaluated. The various strains were purchased from the American Type Culture Collection (ATCC) and amplified in two infections using cell lines A and/or B and upstream conditions A or D [Table 3] using infection protocols common for producing enteroviruses. Cells were planted in tissue culture-treated vented flasks in growth media. Several days post plant, the growth media was decanted and 1 mL of enterovirus inoculum was added to the cell layer. The flasks were incubated for 2 hours before 39 mL of production media was added to each flask and incubated dependent on the upstream condition. The flasks were harvested by collecting the supernatant after visual inspection for cytopathic effect. The flasks were stored at −70° C., thawed, and clarified before use in GSH affinity chromatography purifications.

TABLE 3

| | | | | Pro- | Up- |
|---|---|---|---|---|---|
| | | | | duction | stream |
| | | Abbre- | Enterovirus | Cell | Con- |
| Arm | Enterovirus Serotype | viation | Species | Line | dition |
| 1 | Echovirus 1 | E1 | Enterovirus B | A | A |
| 2 | Rhinovirus 1B | RV1B | Rhinovirus A | B | D |
| 3 | Rhinovirus 35 | RV35 | Rhinovirus B | B | D |
| 4 | Coxsackievirus A 13 | CVA13 | Enterovirus C | A | A |
| 5 | Coxsackievirus A 15 | CVA15 | Enterovirus C | A | A |
| 6 | Coxsackievirus A 18 | CVA18 | Enterovirus C | A | A |
| 7 | Coxsackievirus A 20b | CVA20b | Enterovirus C | B | A |
| 8 | Coxsackievirus A 21 | CVA21 | Enterovirus C | B | A |
| 9 | Coxsackievirus A 21 | CVA21 | Enterovirus C | B | D |

GSH affinity chromatography was performed using Opus Robocolumns containing 0.6 mL of Glutathione Sepharose 4 Fast Flow resin (Repligen) on a Tecan Freedom EVO 150 equipped with an 8-channel liquid handling arm with stainless steel syringe tips and an eccentric robot manipulator arm (Tecan Group Ltd.) in a process described in Konstantinidis et al [17]. For each purification arm, the 0.6 mL columns were equilibrated with 5-CVs of an equilibration solution of Phosphate Buffered Saline (PBS), pH 7.4. The clarified enterovirus sample was applied to the column at a loading of 50-CVs. The column was then washed with 5-CVs of wash 1 solution containing 15 mM Tris, 400 mM NaCl, 1 mM DTT, 0.005% w/v PS80, pH 8.0 and then 5-CVs of wash 2 solution containing 15 mM Tris, 150 mM NaCl, 1 mM DTT, 0.005% w/v PS80, pH 8.0. The bound enterovirus particles were eluted with 5-CV's of an elution solution containing 15 mM Tris, 150 mM NaCl, 1 mM DTT, 1 mM GSH, 0.005% w/v PS80, pH 8.0 through competitive displacement from the immobilized glutathione ligand with free GSH in the mobile phase. The column was then stripped with 5-CVs a strip solution containing 15 mM Tris, 1000 mM NaCl, 1 mM DTT, 10 mM GSH, 0.005% w/v PS80, pH 8.0. All phases were performed at residence times of 4 min, and fractions were collected every ⅓ CV in clear flat bottom 96 w UV plates (Corning).

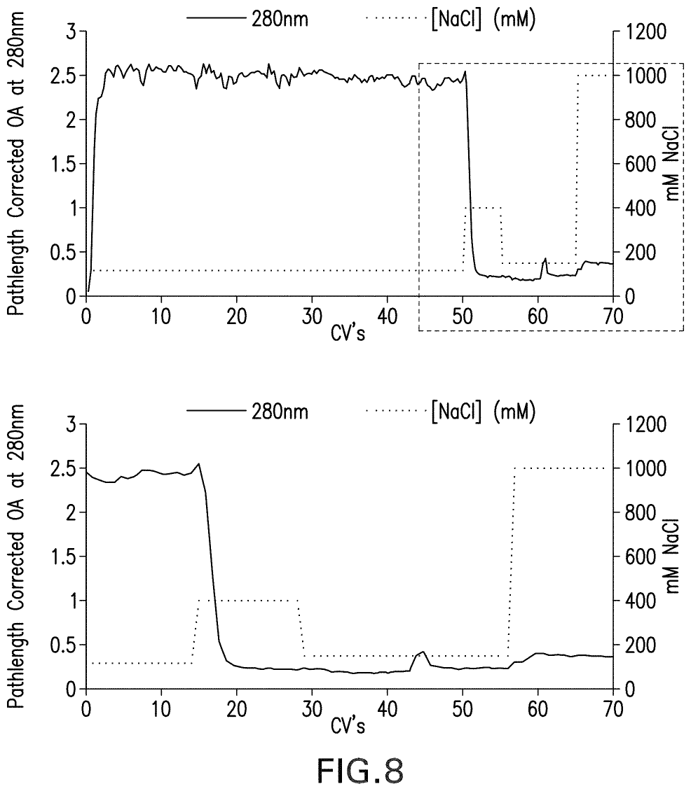

Chromatograms were generated by measuring the optical absorbance of all fractions at 260 nm and 280 nm, pathlength corrected against 900/990 nm, and compiling the pathlength corrected absorbances for all fractions per column. FIG. 8 shows an example chromatogram with the A280 and conductivity traces from purification arm 9. An elution peak was typically observed between 60-65-CVs within the Robocolumn method. The clarified harvest and elution fraction for each purification arm were assayed by SDS-PAGE. Samples were mixed with a 4× loading dye and 10× reducing agent (BioRad) then denatured at 70° C. for 10 min. 25 uL per sample and 2 uL of Mark-12 protein ladder (Invitrogen) were loaded in 1.0 mm 10-lane 12% acrylamide Bis-Tris NuPAGE gels (Invitrogen) and electrophoresed in a gel box and power supply system (Invitrogen) containing 1× MOPS electrophoresis running buffer (Invitrogen) at 200V for 45 min. The gels were then stained using a Pierce silver stain kit (Thermo Fisher Scientific) and imaged using a gel imager (BioRad).

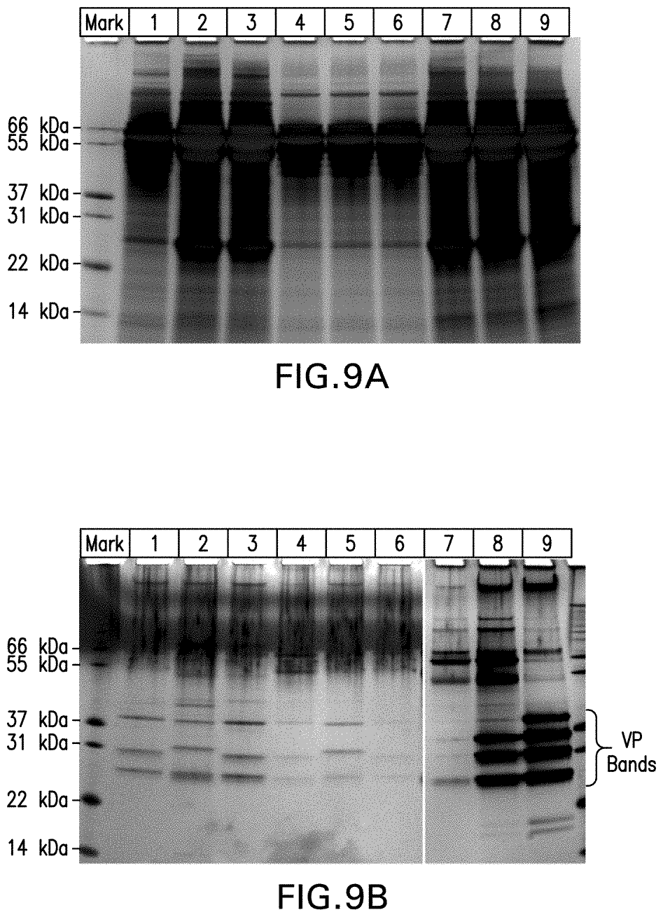

SDS-PAGE analysis of experiment arm 1-9 clarified cell culture harvests [FIG. 9A] and GSH elution fractions [FIG. 9B] demonstrate that GSH affinity chromatography can be used effectively to purify multiple serotypes of enterovirus across a range of different species. Enterovirus capsid viral proteins (VP) are detectable in the elution of Echovirus 1 (1), Rhinovirus 1B (2), Rhinovirus 35 (3), Coxsackievirus A 13 (4), Coxsackievirus A 15 (5), Coxsackievirus A 18 (6), Coxsackievirus A 20b (7), and Coxsackievirus A 21 (8, 9). While all arms used conventional cell culture protocols for enterovirus production, only the CVA21 (8,9) production methods were optimized for high titer. In this example, other enterovirus serotypes (1-7) sourced from ATCC stocks underwent little or no processing from the original vial. Improved cell culture titers and recovery by GSH-affinity chromatography are expected with further optimization of serotype specific methods. Furthermore, the upstream condition may impact the binding of empty procapsids to GSH chromatography resins as evidenced by the different distribution of viral protein bands in the Arm 8-9 GSH elution fractions.

### Example 3

### GSH Affinity Chromatography Purification of CVA21 Using Clarified Harvests Produced With Different Upstream Conditions

GSH affinity chromatography was evaluated across 2 experiments using a similar procedure as in Example 1 with CVA21 clarified harvests produced using upstream cell culture conditions A-C [Table 4]. 20 mL HiPrep columns packed with GSH Sepharose 4 Fast Flow resin were used on an Akta Pure 150M FPLC system with UNICORN system control software. The CVA21 clarified cell culture harvests were loaded to the column at a flow rate of 100 cm/hr until a column loading of 200-CVs was reached. The GSH column was washed at a flow rate of 150 cm/hr with 8-CVs of a GSH Wash 1 buffer containing 15 mM Tris, 400 mM NaCl, 0.005% w/v PS-80, pH 8.0 and then 4-CVs of a GSH Wash 2 buffer containing 15 mM Tris, 75 mM NaCl, 0.005% w/v PS-80, 1 mM DTT, pH 8.0. The bound CVA21 particles were eluted with 4-CVs of a GSH Elution solution containing 15 mM Tris, 75 mM NaCl, 0.005% w/v PS-80, 1 mM DTT, 1 mM GSH, pH 8.0 at a flow rate of 150 cm/hr. The GSH column was stripped with 4-CVs of a GSH Strip buffer containing 15 mM Tris, 1000 mM NaCl, 0.005% w/v PS-80, 1 mM DTT, 10 mM GSH, pH 8.0 and regenerated with a 0.1 N NaOH, 1 M NaCl solution at a flow rate of 150 cm/hr.

TABLE 4

| Experiment | Experiment A | | Experiment B | | |
|---|---|---|---|---|---|
| Experiment | 1 | 2 | 3 | 4 | 5 |
| Arm | | | | | |
| Upstream | B | A | B | C | A |
| Condition | | | | | |

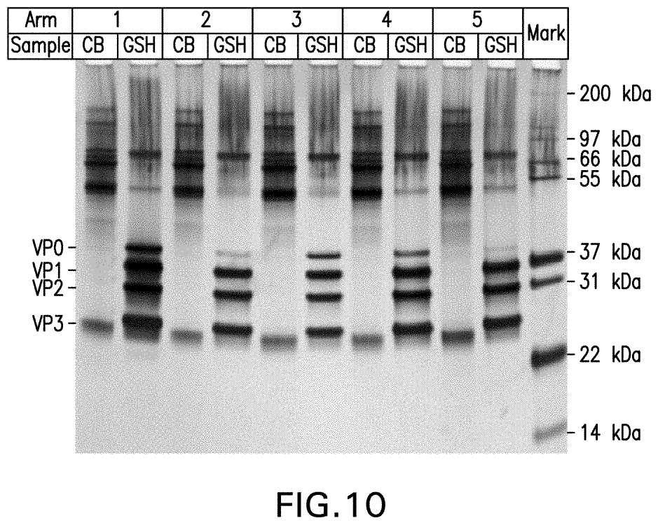

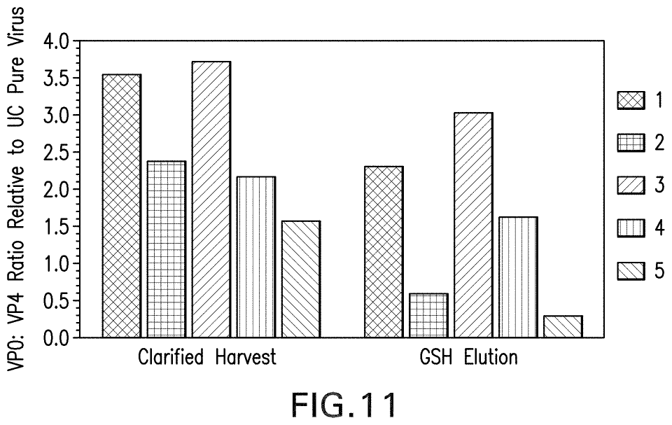

The clarified harvest and GSH elution product samples were analyzed by SDS-PAGE with silver stain [FIG. 10] and capillary electrophoresis anti-VP4 western to detect VP0:VP4 ratio relative to an ultracentrifugation purified virus [FIG. 11]. Analysis of SDS-PAGE showed the impurity protein clearance in the GSH elution was similar for all arms, but the GSH elution samples had different intensities of the VP0 band (˜37 kDa) relative to the other viral protein bands. Arm 1, 3, and 4 had higher VP0 content, while Arm 2 and 5 had low VP0 content, indicating differences in empty procapsid clearance across the GSH chromatography step. The empty procapsid:full mature virus particle ratio relative to an ultracentrifugation purified virus by anti-VP4 western demonstrated that the VP0:VP4 ratio decreased from the clarified harvest to the GSH elution product for all arms, but there were differences in the reduction factor. In Arms 2 and 5, which was produced from upstream condition A, the empty procapsid:full mature virion ratio was significantly less than that of ultracentrifugation. These results demonstrate that under some upstream conditions, a fraction of empty procapsids may bind to GSH resin, and a second step such as cation exchange (CEX) chromatography may be implemented to clear the residual empty procapsids in the GSH elution product to meet or exceed the purity of an ultracentrifugation purified virus.

### Example 4

### Purification of Enterovirus Using a Process Involving GSH Affinity Chromatography and CEX Chromatography

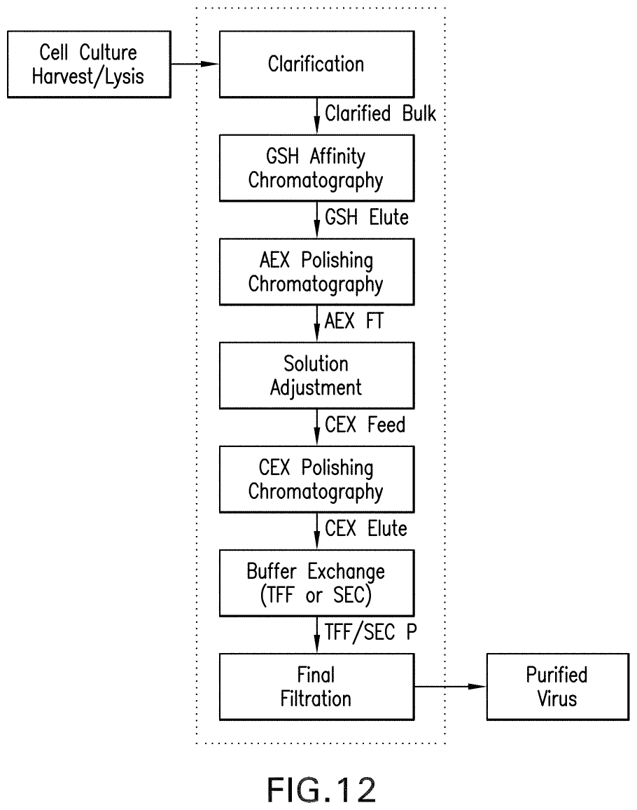

A scalable purification of enteroviruses was demonstrated using the process in FIG. 12 with CVA21 purification from a large-scale bioreactor cell culture harvest as an example. The purification process involves the harvest of enterovirus cell culture consisting of cell culture media, host cell debris, serum impurities, and enterovirus particles through one or multiple clarification filters with a pore size range of 0.2-100 μm to remove host cell debris. A series of two clarification steps may be used with a primary clarification step with a filter pore size of 1-100 μm and a secondary clarification step with a filter pore size of 0.2-5 μm. For harvests from microcarrier cell culture, the primary clarification may involve a mesh bag or a depth filter to remove microcarriers prior to the secondary clarification. In the current example with CVA21, the clarification step was run continuously with 2 filters in series operated at 100 L/m²-hr (LMH): Primary clarification with a Clarisolve 60 HX (Millipore) 60 μm depth filter to remove microcarriers and large cell debris, and a secondary clarification with a Sartopure GF+ (Sartorius) 1.2 μm depth filter to clear smaller cell debris including HC-DNA.

In some enterovirus cell cultures, the lytic activity of the virus is sufficient to lyse the cells and no lysis step is needed. In other enterovirus cell cultures, a lysis step such as detergent lysis with PS-80, PS-20, or other surfactant ranging from 0.01-2% w/v may be implemented prior to the clarification step to fully lyse the cells. In the current example with CVA21, no lysis step was performed.

The clarified harvest is loaded directly to a GSH affinity chromatography column following a similar procedure as in Example 1. For the GSH chromatography operation, GSH immobilized resin is packed into manufacturing scale chromatography columns and operated with a chromatography skid such as Akta Pilot (Cytiva) or Akta Ready (Cytiva). In the current example with CVA21, a 14 cm diameter column packed with GSH Sepharose 4 FF was used on the Akta Pilot with UNICORN system control software. The CVA21 clarified cell culture harvest was loaded to the column at a flow rate of 100 cm/hr until a column loading of 150-200 CVs. The GSH column was washed at a flow rate of 150 cm/hr with 8-CVs of a GSH Wash 1 buffer containing 15 mM Tris, 400 mM NaCl, 0.005% w/v PS-80, pH 8.0 and then 4-CVs of a GSH Wash 2 buffer containing 15 mM Tris, 150 mM NaCl, 0.005% w/v PS-80, 1 mM DTT, pH 8.0. The bound CVA21 particles were eluted with 4-CVs of a GSH Elution solution containing 15 mM Tris, 150 mM NaCl, 0.005% w/v PS-80, 1 mM DTT, 1 mM GSH, pH 8.0 at a flow rate of 150 cm/hr. The GSH column was stripped with 4-CVs of a GSH Strip buffer containing 15 mM Tris, 1000 mM NaCl, 0.005% w/v PS-80, 1 mM DTT, 10 mM GSH, pH 8.0 and regenerated with a 0.1 N NaOH, 1 M NaCl solution at a flow rate of 150 cm/hr.

The GSH elution product is loaded directly to an optional polishing anion exchange (AEX) chromatography step operated in flow-through mode for additional residual impurity clearance. The AEX chromatography step may use common AEX chromatography media such as POROS 50HQ (ThermoFisher), Capto Q (Cytiva), or Nuvia Q (BioRad) or other AEX stationary phases. For large scale AEX chromatography operation, AEX resin is packed into manufacturing scale chromatography columns and run with a chromatography skid such as Akta Pilot at a flow rate of 50-300 cm/hr. The AEX column is equilibrated in 3-5 CV AEX Equilibration buffer composed of a solution at pH 6-9 and a monovalent salt concentration of 50-500 mM. The GSH elution product in a solution at pH 6-9 and a monovalent salt concentration of 50-500 mM is loaded to the AEX column followed by a 1-3 CV chase with the AEX equilibration buffer. The enterovirus particles flow through while impurities including HC-DNA and impurity protein bind to the AEX resin. The column is stripped with 3-5 CV AEX Strip buffer composed of a solution at pH 6-9 and a monovalent salt concentration of 500-1500 mM and regenerated with a solution containing 0.1-0.5 N sodium hydroxide. The AEX buffer solutions may contain a surfactant such as PS-80, PS-20 or other similar surfactant at a concentration of 0.001-1% w/v. In the current example with CVA21, a 5 cm diameter column packed with POROS 50HQ resin was run on an Akta Pilot with UNICORN system control software at a flowrate of 200 cm/hr. The AEX column was equilibrated with 4-CV of an AEX equilibration buffer consisting of 15 mM Tris, 150 mM NaCl, 0.005% w/v PS-80, pH 8.0. The GSH elution product containing CVA21 particles was loaded to the column until a loading of 25-30 CVs and chased with 2-CV AEX equilibration buffer. The CVA21 particles flowed through while residual impurities bound to the column. The AEX column was stripped with 4-CVs of an AEX Strip buffer containing 15 mM Tris, 1000 mM NaCl, 0.005% w/v PS-80, pH 8.0 and regenerated with 4-CVs of a 0.5 N NaOH solution. The AEX chromatography step may be omitted if the desired residual impurity specifications in the final purified composition are met without AEX. In this situation, the GSH elution product is forwarded to the solution adjustment step.

In the solution adjustment step, the AEX FT or GSH elution (if AEX is not performed) product is adjusted to solution conditions compatible with binding to the CEX chromatography resin in the subsequent CEX chromatography step. The AEX FT or GSH elution product is initially in a solution at pH 6-9 and a monovalent salt concentration of 50-500 mM. If necessary, concentrated stock solutions of 0.5-1.5 M adjustment buffer solution, consisting of a buffer species such as citrate, at pH 3.5-6.0 and 2-5 M adjustment monovalent salt solution, such as NaCl, are spiked into the AEX FT to bring the solution pH down to pH 3.5-6.0 and increase the monovalent salt concentration to 50-500 mM. One or both adjustment solutions may not be required if the AEX FT is already at the target pH or monovalent salt concentration of the loading solution to the CEX step. In the current example with CVA21, a 1M sodium citrate, pH 4.0 solution and a 5 M NaCl solution are spiked into the AEX FT, initially at pH 8.0 and 150 mM NaCl, to target a final sodium citrate concentration of 50 mM at pH ˜4.1 and a final NaCl concentration of 400 mM. The concentrated stock solutions were slowly added to the AEX FT product over 5-10 minutes with mixing. This solution adjusted sample was designated CEX feed and represented the target CEX loading solution.

The CEX chromatography step, operated in bind-elute mode, is implemented to improve process robustness as a secondary step for empty procapsid clearance, to clear residual impurities, and to provide additional volume reduction. The CEX step may use common chromatography media such as POROS 50HS (ThermoFisher), Capto S (Cytiva), or Nuvia S (BioRad) or other CEX stationary phases. For large scale CEX chromatography operation, CEX resin is packed into large scale chromatography columns and run with a chromatography skid such as Akta Pilot at a flow rate of 50-300 cm/hr. The CEX column is equilibrated in 3-5 CV CEX Equilibration buffer composed of a solution at pH 3.5-6.0 and a monovalent salt concentration of 50-500 mM. The CEX feed in a CEX loading solution at pH 3.5-6.0 and a monovalent salt concentration of 50-500 mM is loaded to the CEX column. The enterovirus particles bind to the CEX resin while some residual impurities may flow through. The CEX column is washed with 3-5-CVs of a CEX Wash buffer solution, composed of a solution at pH 3.5-6.0 and a monovalent salt concentration of 100-600 mM, to remove residual impurities. The full mature virions are selectively eluted from the CEX column using 3-5-CVs of a CEX elution buffer solution, composed of a solution at pH 3.5-4.8 and a monovalent salt concentration of 200-1000 mM NaCl, while the empty procapsids remain bound to the CEX resin. The empty procapsids and other residual impurities are eluted with 3-5 CV CEX Strip buffer, composed of a solution at pH 4.0-8.0 and a monovalent salt concentration of 500-1500 mM and the CEX column is regenerated with a solution containing 0.1-0.5 N sodium hydroxide. The CEX buffer solutions may contain a surfactant such as PS-80, PS-20 or other similar surfactant at a concentration of 0.001-1% w/v. In the current example with CVA21, a 5 cm diameter column packed with POROS 50HS resin was run on an Akta Pilot with UNICORN system control software at a flowrate of 200 cm/hr. The CEX column was equilibrated with 4-CVs of an CEX equilibration buffer consisting of 50 mM sodium citrate, 400 mM NaCl, 0.005% w/v PS-80, pH 4.0. The CEX feed product containing CVA21 particles was loaded to the column until a loading of 25-30 CVs. The column was washed with 4-CVs of a CEX Wash buffer consisting of 25 mM sodium citrate, 500 mM NaCl, 0.005% w/v PS-80, pH 4.0. The full mature CVA21 virions were selectively eluted from the CEX column with 4-CVs of a CEX elution buffer consisting of 25 mM sodium citrate, 800 mM NaCl, 0.005% w/v PS-80, pH 4.0. The empty CVA21 procapsids were eluted with 4-CVs of a CEX strip buffer consisting of 25 mM sodium citrate, 1000 mM NaCl, 0.005% w/v PS-80, pH 7.0 and the column was regenerated with 4-CVs of a 0.5 N NaOH solution.

The CEX elution product, consisting of purified full mature enterovirus virions, is buffer exchanged into a stabilizing buffer by ultrafiltration/diafiltration (UF/DF) via tangential-flow filtration (TFF) or size-exclusion chromatography (SEC) in desalting mode. For TFF, the enterovirus particles are retained by a hollow fiber or a cassette with a molecular weight cut-off of about 50-500 kDa, while other small solution components permeate through the membrane. The TFF may be operated with a crossflow shear rate of about 1,000-8,000 s⁻¹, a transmembrane pressure (TMP) of about 0.1-10 psig, and a permeate flux of about 5-60 L/m²-hr. The CEX elution product is diafiltered with 5-10 diavolumes into a lx stabilizing buffer solution consisting of a buffering species at about pH 6-8. A UF step may be performed before or after DF. An optional neutralization step may be performed prior to TFF where the CEX elution product is diluted 2-5-fold into a 2-5× concentrated stabilizing buffer solution. An optional filtration step consisting of a filter with a pore size of about 0.1-1 μm may be used prior to TFF. For buffer exchange with SEC, the CEX elution product is loaded to SEC column packed with resin such as Sephadex (Cytiva) and operated in desalting mode using a chromatography skid such as Akta Pilot. In the current example with CVA21, the CEX elution product was neutralized by diluting 3-fold into a 3× concentrated stabilizing buffer solution. The neutralized CEX elution product was filtered using a Durapore 0.22 μm filter (Millipore) prior to generate a TFF feed solution. The TFF feed solution was initially concentrated 2-3-fold and then buffer exchanged into the 1× stabilizing buffer solution using a Spectrum 300 kDa hollow fiber filter (Repligen) at a crossflow of 2000 s⁻¹, TMP of 1-2 psig, and permeate flux of 20-40 LMH.

A final filtration step is performed with the buffer exchanged TFF or SEC elution product. A filter pore size of 0.1-0.5 μm is used. The final purified composition of enterovirus in the stabilizing buffer solution is frozen and stored at <−60° C. In the current example with CVA21, a Durapore 0.22 μm filter (Millipore) was used.

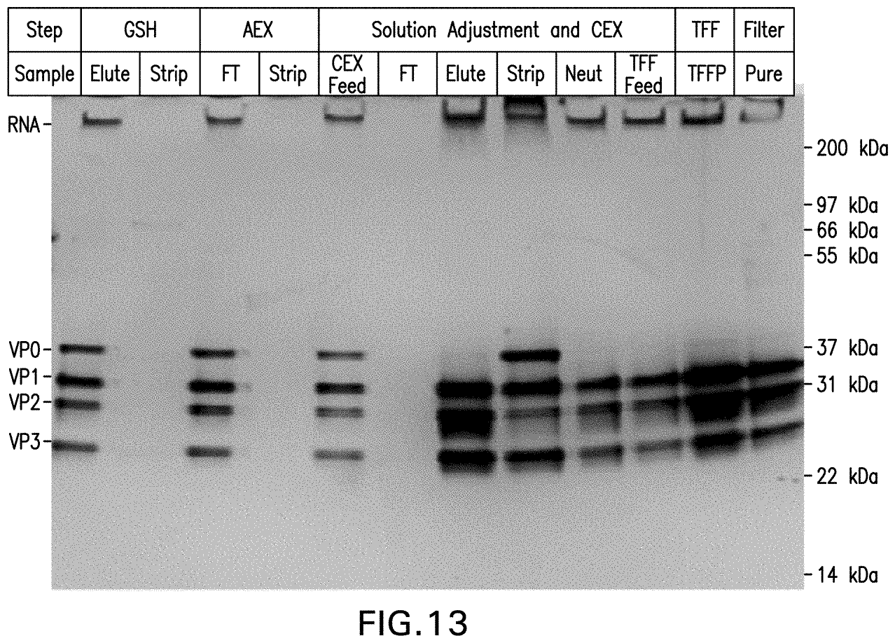

The CVA21 purification process detailed above was demonstrated for 4 batches produced from upstream cell culture conditions A and B [Table 6]. As an example, the purification process intermediate samples for Batch 4 with cell culture condition B were characterized by SDS-PAGE with silver stain [FIG. 13]. The GSH elution product demonstrated high purification of residual protein impurities with only VP0, VP1, VP2, VP3 (VP4, 7 kDa, ran off gel), and RNA detectable bands. The combination of VP0 and VP2 content indicated the GSH elution product contained a distribution of empty procapsid and mature virions. Trace amounts of residual impurities were cleared in the AEX Strip and CEX FT. The CEX elution product had a high concentration of only VP1, VP2, VP3, and RNA bands visible, confirming the clearance of empty procapsids and a pure composition of full mature virions. The empty capsids were eluted in the CEX strip sample, evidenced by the high VP0 content. The VP band distribution remained constant after the CEX elution product was neutralized and filtered prior to the TFF buffer exchange and final filtration steps. The plaque infectivity, RT-qPCR genome, and HPSEC particle (see analytical method in Example 6) step yields across the GSH and CEX chromatography steps confirmed a high yielding purification process [Table 5]. These results demonstrate a robust production of a purified composition of mature virions involving multiple unit operations capable of removing empty procapsids (GSH, CEX), clearing residual impurities (GSH, AEX, CEX), and reducing process volume (GSH, CEX, TFF).

TABLE 5

| Batch 4 | Plaque | RT-qPCR | HPSEC |
|---|---|---|---|
| Step Yields | Infectivity | Genomes | Particles |
| Clarified Harvest to GSH | 85% | 84% | n/a |
| Elute Step Yield | | | |
| GSH Elute to CEX Elute | 83% | 87% | 77% |
| Step Yield | | | |

### Example 5

### Purified Compositions of CVA21 Produced From 4 Large Scale Batches Involving GSH Affinity Chromatography and CEX Chromatography Compared to an Ultracentrifugation Purified CVA21 Composition

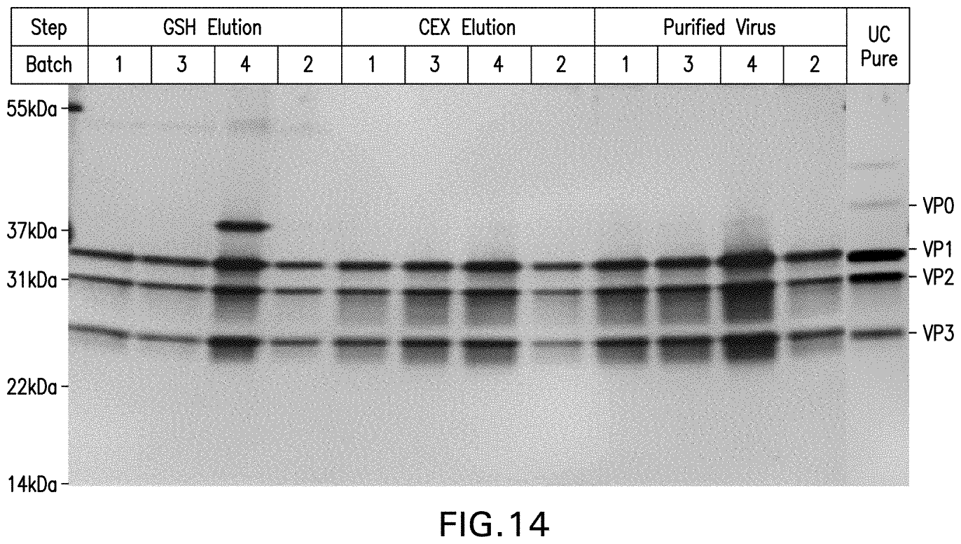

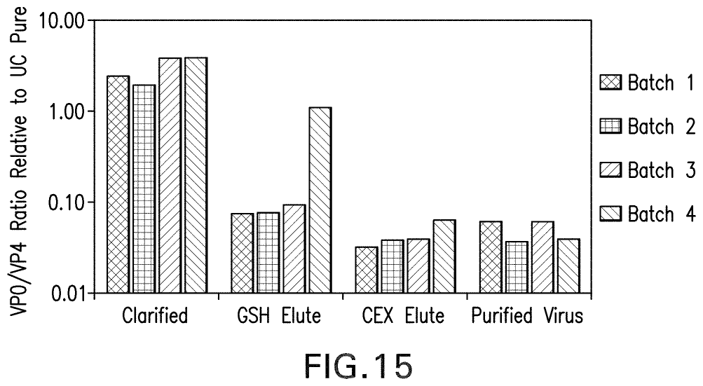

Four purified compositions of CVA21 generated from cell culture condition A and B and purified using the process detailed in FIG. 13 as well as a purified virus produced using ultracentrifugation (UC Pure, FIG. 1A) were characterized by SDS-PAGE [FIG. 14], capillary electrophoresis anti-VP4 western [FIG. 15], and several assays including plaque potency, genome RT-qPCR, HPSEC, RP-HPLC, CE-SDS, HC-DNA qPCR, and BSA ELISA [Table 6]. See analytical methods in Example 6 for detailed descriptions of each assay. A commercially available BSA ELISA kit (Cygnus) was used for BSA analysis.

TABLE 6

| Purified | | Plaque | RT-qPCR | HPSEC | RP-HPLC | CE-SDS | qPCR | ELISA |
|---|---|---|---|---|---|---|---|---|
| Virus | Upstream | Potency | Genomes | Particles | VP0:VP2 | VP Purity | HC-DNA | Resid BSA |
| Batch | Condition | (pfu/mL) | (gen/mL) | (part/mL) | Peak Ratio | (% Area) | (ng/mL) | (ng/mL) |
| Batch 1 | A | 4.09E+09 | 1.91E+12 | 1.60E+12 | 0.12% | >95% | <0.02 | <5 |
| Batch 2 | A | 2.77E+09 | 1.46E+12 | 1.39E+12 | 0.19% | >95% | <0.02 | <5 |
| Batch 3 | A | 2.90E+09 | 1.76E+12 | 1.61E+12 | 0.16% | >95% | <0.02 | <5 |
| Batch 4 | B | 1.13E+10 | 5.67E+12 | 3.10E+12 | n/a | >95% | <0.02 | <5 |
| UC Pure | n/a | 1.21E+09 | 1.14E+12 | 7.80E+11 | 3.1% | >95% | 0.755 | <5 |

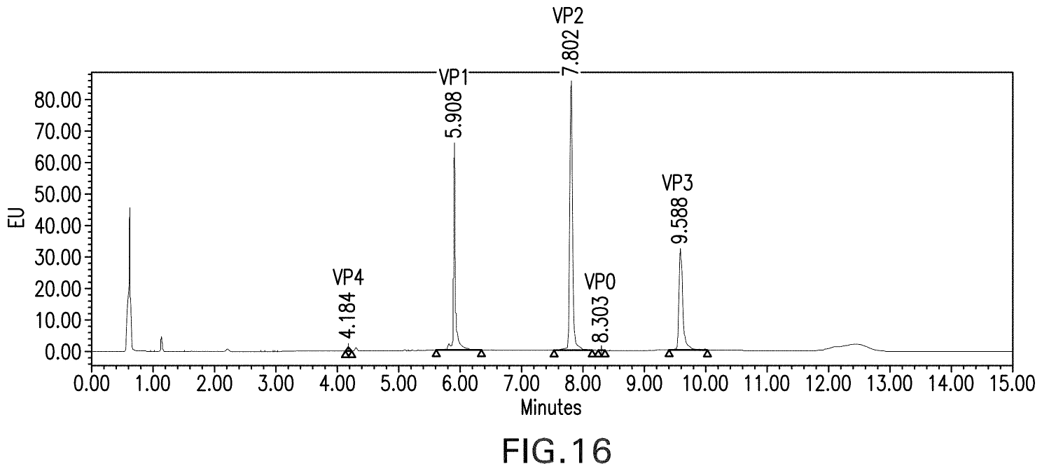

Analysis of the SDS-PAGE with silver stain and anti-VP4 western demonstrates empty procapsid clearance for Batches 1-3 in the GSH elution product, while Batch 4 has high VP0 content relative to UC pure virus. However, the CEX step clears the empty procapsids and in all batches, the empty procapsids:full mature virus ratios were ˜10× lower than the UC pure CVA21 in the CEX elution product in the final purified virus composition. Furthermore, the reverse-phase HLPC (RP-HPLC) assay with H-Class BIOshell C4 column (Waters) was used to evaluate the VP0-containing particle:VP2-containing particle ratio of the purified virus samples by measuring a ratio of VP0:VP2 peak area signals from an average of 2 injections, using the Batch 1 chromatogram as an example [FIG. 16]. The RP-HPLC results of Batches 1-3 were ˜10× lower than the UC purified virus, corroborating the anti-VP4 western analysis.

The plaque potency, RT-qPCR genomes, and HPSEC particle concentrations of the purified composition of Batch 1-4 were higher than the UC pure due to improved process titer and yields. The SDS-PAGE, and CE-SDS assays demonstrate high protein purity in Batch 1-4, and the HC-DNA and BSA assays were all <LOQ (Limit of Quantification) and demonstrate improved HC-DNA clearance compared to the UC pure composition. With an assumed dose of 5E+07 pfu/dose, Batch 1-4 achieved <1 pg HC-DNA/dose and <100 pg BSA/dose, several orders of magnitude lower than the typical guidance of <10 ng HC-DNA/dose and <50 ng BSA/dose [Table 7].

TABLE 7

| Purified Virus | HC-DNA per dose | BSA per dose |
|---|---|---|
| Batch | (pg DNA/dose) | (pg BSA/dose) |
| Batch 1 | <0.2 | <61 |
| Batch 2 | <0.4 | <90 |
| Batch 3 | <0.3 | <86 |
| Batch 4 | <0.1 | <22 |
| UC Pure | 31.2 | <206 |

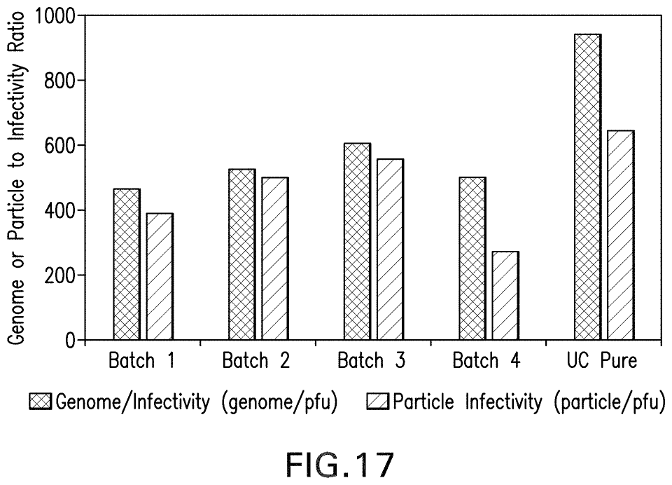

Total particle and genome to infectious particle ratios are important product attributes to track for process consistency and purified virus quality. The total to infectious particle ratio may range from 1:1 to 10⁷:1 and depends on the individual virus and analytical assays used in the calculation of the value [18]. The genome and particle to infectivity ratios in genome/pfu and particle/pfu, respectively for Batch 1-4 were lower than the UC pure virus, signifying improved product quality [FIG. 17]. These analytical results confirm a more robust and scalable process capable to producing CVA21 compositions of improved viral potency, mature virion purity, and residual impurity clearance relative to existing compositions produced by conventional methods of ultracentrifugation or other methods.

### Example 6

### CVA21 Purified Virus Analytical Assays

### Reverse Phase HPLC or UPLC Assay Procedure

A general HPLC or UPLC system with UV and Fluorescence (FLR) detector is suitable for use to separate CVA21 capsid proteins under reverse phase chromatographic conditions. A typical chromatographic system consists of a Waters ACQUITY UPLC System, including a quaternary (or binary) pump, sample manager, column component, FLR detector and TUV Detector. A typical column for use is Millipore (Sigma-Aldrich) BIOshell IgG C4 column with 1000 ø pore size and 2.7 μm particle size. A 2.1 ×100 mm size column (Catalog 63288-U) is sufficient for separation of all capsid virion proteins. Columns with different size or with similar sizes from other vendors could achieve equivalent separation efficiency. A typical column temperature is maintained at 80° C., but a column temperature range from 65-85° C. may be used without observable impact on virion protein separation. FLR detection is recorded using excitation at 280 nm and emission at 352 nm. Dual UV detections are recorded at both 220 nm and 280 nm. In some cases, UV detection at 260 nm is also recorded. Sample manager temperature is maintained at around 8° C. during analysis.

Two mobile phases are used for gradient elution. The first mobile phase consists of 0.1% trifluoroacetic acid (TFA) in HPLC-grade water (Mobile Phase A); the second mobile phase is 0.1% TFA in acetonitrile (Mobile Phase B). The mobile phase gradient and flowrate used for analysis of CVA21 purified virus samples is depicted in Table 8. The viral protein peaks identified in the example in FIG. 16 were confirmed by mass spectrometry. The gradient settings may be modified, depending on the sample injected, and flow rates from 0.2 to 0.5 mL/min may be used, depending on the system pressure limits. The viral protein peak retention time may shift depending on the system, column, and method, but the relative peak order is expected to remain the same. About 5-50 μL CVA21 purified samples are injected directly without pre-dilution or reduction for HPLC analysis. If a sample is too dilute (less than about 0.1 μg injection) or below the limit of detection of an analytical method, the sample may be concentrated using centrifugal filter concentrators such as Amicon (Millipore) or Vivaspin (Sartorius) filters into the ideal range of the assay.

TABLE 8

| | Flow Rate | % Mobile | % Mobile |
|---|---|---|---|
| Time | (mL/min) | Phase A | Phase B |
| 0 | 0.4 | 75 | 25 |
| 0.5 | 0.4 | 75 | 25 |
| 4.5 | 0.4 | 60 | 40 |
| 10.5 | 0.4 | 54 | 46 |
| 11.0 | 0.4 | 20 | 80 |
| 11.5 | 0.4 | 75 | 25 |
| 12.5 | 0.4 | 75 | 25 |

### CE-SDS Assay Procedure

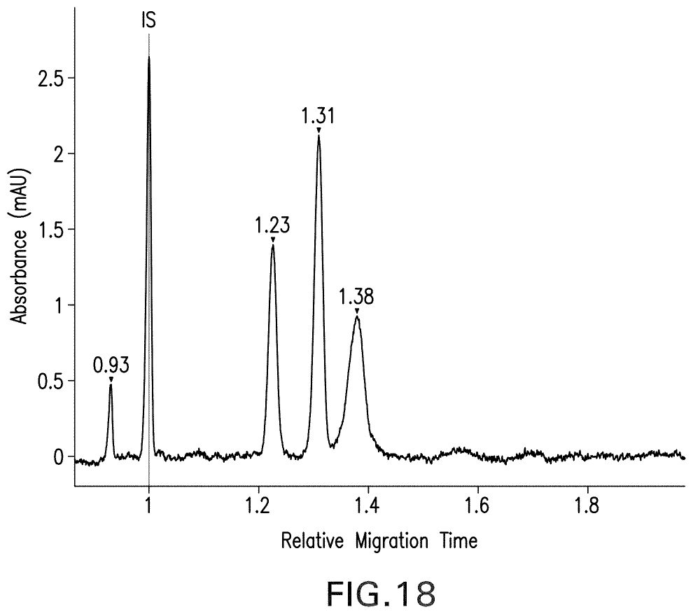

The CVA21 mature virion capsid is composed of 4 viral proteins, VP4, VP3, VP2 and VP1. CE-SDS is used to separate, identify, and quantify the 4 VPs based on their different molecular weights and obtain a relative % peak area and relative migration time. The assay can also detect VP0, which is a marker for empty procapsids. The CE-SDS loading solution is prepared by mixing 50 μL of CVA21 sample with 50 μL of a master mix composed of 47 μL of 1× sample buffer, 2 μL of 2-mercaptoethanol and 1 μL of 10× internal standard (10 kDa protein) using Maurice CE-SDS Plus kit reagents (Protein Simple). The sample is heated at 70° C. for 10 min, placed on the benchtop for 1 min to cool down, and then vortexed at 1,000 g for 1 min. The loading solution is transferred into a 96 well plate by pipetting 50 uL in the assigned wells and the plate was centrifuged at 1,000 g for 5 min using a centrifuge plate adapter. The sample plate is placed in the Maurice Instrument (Protein Simple) with the Maurice CE-SDS Plus kit separation cartridge (Protein Simple) and 50 μL of the loading solution is injected at 4600V for 120 sec and separated at 5750V for 35 min. A typical electropherogram (with about a 10 μg injection), showing the separation of the 4 VPs of the purified virus sample from Batch 4 is shown in FIG. 18. The viral protein expected relative migration times and % peak area is shown in Table 9. The expected relative migration times may be shifted by up to 5% for a given separation. The % VP purity is calculated by the summation of the % peak areas of VP1-4 with a detection limit of quantitation of around 5%. If a sample is too dilute (less than about 1 μg injection) or below the limit of detection of an analytical method, the sample may be concentrated using centrifugal filter concentrators such as Amicon (Millipore) or Vivaspin (Sartorius) filters into the ideal range of the assay.

TABLE 9

| | Expected Relative | Example Batch 4 |
|---|---|---|
| Viral Protein | Migration Time | % Peak Area |
| VP4 | 0.93 | 8% |
| VP3 | 1.23 | 26% |
| VP2 | 1.31 | 37% |
| VP1 | 1.38 | 29% |
| VP0 | 1.39 | <5% |

### HPSEC Assay Procedure

The HPSEC assay is performed on a Agilent Series 1260 or higher. The system consists of a plate autosampler for injection and a quaternary pump. The HPSEC assay procedure is conducted using a TSKgel G5000PW×1 (7.5×300 mm, 17 μm) column obtained from Tosoh Bioscience LLC (Cat#0005764). The system is calibrated using Bovine Serum Albumin (Pierce). A mobile phase containing 10 mM Bis-Tris, 0.6 M NaCl, pH 6.9 is used to equilibrate the HPSEC system and CVA21 samples are injected (greater than about 1 μg) and resolved at a flow rate of 0.4 mL/min under isocratic elution. The total elution time following sample injection is 35 min at 30° C. A DAD UV detector is used to acquire absorbance at 280 nm. Wyatt Astra-7 software converts UV A₂₈₀ peak area to virus mass directly, virus concentration is calculated following the equation shown below:

Virus concentration (μg/mL)=virus mass (μg)/injection volume (mL)

The CVA21 full mature virion extinction coefficient (=5.48) at 280 nm was calculated based on RNA sequence and amino acid sequence of all viral proteins, using an informatics tool with algorithm [Protein Identification and Analysis Tools on the ExPASy Server, E. Gasteiger, C. Hoogland, A. Gattiker, S. Duvaud, M. R. Wilkins, R. D. Appel, A. Bairoch; The Proteomics Protocols Handbook, Humana Press 2005 (Informatics tool: https://web.expasy.org/protparam); or Determination of Extinction Coefficient, V. Murugaiah, Handbook of Analysis of Oligonucleotides and Related Products 2011 (Informatics tool: “Quest Calculate™ RNA Molecular Weight Calculator.” AAT Bioquest, Inc, 6 May. 2020, https://www.aatbio.com/tools/calculate-RNA-molecular-weight-mw]. To obtain the viral particle concentration, the particle number was related to the injection volume. Equation to calculate the viral particle counts is shown below:

Particle count (per mL)=[virus concentration (g/mL)/*Mw (Da)]×Avogadro constant (6.02E+23 particles-mol⁻¹)

*CVA21 full mature virus (capsid proteins+RNA) Mw (8203 Kda) was calculated using protein and RNA sequence of MVSS01 in Example 7

If a sample is too dilute (less than about 1 μg injection) or below the limit of detection of an analytical method, the sample may be concentrated using centrifugal filter concentrators such as Amicon (Millipore) or Vivaspin (Sartorius) filters into the ideal range of the assay.

### Plaque Assay Procedure

The plaque assay determines the infectivity (pfu/mL) of CVA21 after infection of SK-Mel-28 cells. In this method, SK-Mel-28 cells are seeded in 12-well cell culture plates as 5.0E+05 cells/well and incubated at 37° C., 5% CO₂ for 24±4 hours. The cells are then infected with CVA21 samples and incubated at 37° C., 5% CO₂ for 90 to 105 minutes to allow for virus adsorption. Following the adsorption period, an overlay (1% Methyl cellulose as 1.5 mL/well) is added, and the infected cell plates are returned to the incubator for 72±2 hours. After this incubation, the overlay is removed, cell plates are washed twice with PBS/DPBS and fixed with 80% methanol. The plates are then stained with Coomassie brilliant blue staining solution which binds to all adherent cells that are uninfected. Infected cells will not adhere to the plate surface and leave a gap which is designated as a plaque. Plaques on the plates are visibly counted manually with a light box. Titer calculations are performed by multiplying the number of plaques with the respective dilution factor and represented as plaques/mL. A geomean of at least two wells having plaque numbers between the range of about 5 to 55 plaques per well are used for this calculation.

### RT-qPCR Viral Genome Quantification Assay (GQA) Procedure

Samples are lysed by proteinase K/sodium dodecyl sulfate digestion. Nucleic acids are then extracted by phenol:chloroform:isoamyl alcohol separation and sodium acetate/isopropanol precipitation, followed by an ethanol wash and resuspension in water. Resuspended nucleic acids are added to a 1-step quantitative reverse transcription PCR (RT-qPCR) reaction containing primers and dual-labeled probe targeting the CVA21 VP1 gene, designed against GenBank accession AF465515.1. Amplification is monitored by increase in fluorescence across amplification cycles. Genome copy number is determined by interpolation against a standard curve of a synthetic RNA containing the VP1 gene target region, ranging from 1E+11 genome copies/mL to 1E+07 genome copies/mL.

### Residual Host Cell DNA Assay Procedure

Samples are lysed by proteinase K/sodium dodecyl sulfate digestion. Nucleic acids are then extracted by phenol:chloroform:isoamyl alcohol separation and sodium acetate/isopropanol precipitation, followed by an ethanol wash and resuspension in water. Resuspended nucleic acids are added to a quantitative PCR (qPCR) reaction containing primers and dual-labeled probe targeting the 3′ conserved region of the human LINE-1 transposase domain, designed against NCBI reference accession NM_001164835.1. Amplification is monitored by increase in fluorescence across amplification cycles. Host cell DNA concentration is determined by interpolation against a standard curve of purified MRC-5 DNA, ranging from 2E+02 ng DNA/mL to 2E-02 ng DNA/mL.

Virus TCID₅₀ assay

Confluent monolayers of SK-Mel-28 cells in 96-well tissue culture plates were inoculated with 10-fold serial dilutions (100 μL/well in quadruplicate) of CVA21 and incubated at 37° C. in a 5% CO₂ environment for 72 h. The mouse serum was serially diluted 10-fold ranging from 1:10² to 1:10⁸ in DMEM containing 2% fetal calf serum (FCS). Wells were scored for cytopathic effects (CPE) visually under an inverted microscope. Wells that had detectable CPE were scored positive and the 50% viral endpoint titer was calculated using the Karber method (Dougherty 1964).

### Example 7

### CVA21 Virus Sequence

The prototype Kuykendall strain of Coxsackievirus A21 is described in Genbank as AF465515. The initial clinical trial batch MelTrial Virus 1 was derived from the prototype strain above by plaque purification, expansion in SK-MEL-28 cells and purification by sucrose gradient.

The master virus seed stock CVA21 MVSS-01 used in the above examples was derived from MelTrial Virus 1 by plaque purification and expansion in MRC-5 cells. The complete genomic sequence of this virus was analyzed [Table 10].

TABLE 10
RNA Sequence of MVSS-01

5′-UUAAAACAGCUCUGGGGUUGUUCCCACCCCAGAGGCCCACGUGGCGGCUAG
UACUCUGGUAUUACGGUACCUUUGUACGCCUGUUUUGUAUCCCUUCCCCCGUAACUUUAG
AAGCUUAUCAAAGGUUCAAUAGCAGGGGUACAAACCAGUACCUCUACGAACAAGCACUUC
UGUUUCCCCGGUGAUAUCACAUAGACUGUACCCACGGUCAAAAGUGAUUGAUCCGUUAUC
CGCUUGAGUACUUCGAGAAGCCUAGUAUCACCUUGGAAUCUUCGAUGCGUUGCGCUCAAC
ACUCUGCCCCGAGUGUAGCUUAGGCUGAUGAGUCUGGGCACUCCCCACCGGCGACGGUGG
CCCAGGCUGCGUUGGCGGCCUACCCAUGGCUGAUGCCGUGGGACGCUAGUUGUGAACAAG
GUGUGAAGAGCCUAUUGAGCUACUCAAGAGUCCUCCGGCCCCUGAAUGCGGCUAAUCCUA
ACCACGGAGCAACCGCUCACAACCCAGUGAGUAGGUUGUCGUAAUGCGUAAGUCUGUGGC
GGAACCGACUACUUUGGGUGUCCGUGUUUCCCUUUAUAUUCAUACUGGCUGCUUAUGGU
GACAAUUUACAAAUUGUUACCAUAUAGCUAUUGGAUUGGCCACCCAGUAUUGUGCAAUA
UAUUUGAGUGUUUCUUUCAUAAGCCUUAUUAACAUCACAUUUUUAAUCACAAUAAACAG
UGCAAAUGGGGGCUCAAGUUUCAACGCAAAAGACCGGUGCGCACGAGAAUCAAAACGUG
GCAGCCAAUGGAUCCACCAUUAAUUACACUACUAUCAACUAUUACAAAGACAGUGCGAGU
AAUUCCGCUACUAGACAAGACCUCUCCCAAGAUCCAUCAAAAUUCACAGAACCGGUUAAG
GACUUAAUGUUGAAAACAGCACCAGCUCUAAACUCGCCUAACGUGGAAGCAUGUGGGUA
CAGUGACCGUGUGAGGCAAAUCACUUUAGGCAACUCGACUAUUACUACACAAGAAGCAGC
CAAUGCUAUUGUUGCUUACGGUGAAUGGCCCACUUACAUAAAUGAUUCAGAAGCUAAUC
CGGUAGAUGCACCCACUGAGCCAGAUGUUAGUAGCAACCGGUUUUACACCCUAGAAUCGG
UGUCUUGGAAGACCACUUCAAGGGGAUGGUGGUGGAAGUUACCAGAUUGUUUGAAGGAC
AUGGGAAUGUUUGGUCAGAAUAUGUACUAUCACUACUUGGGGCGCUCUGGUUACACCAU
UCAUGUCCAGUGCAACGCUUCAAAAUUUCACCAAGGGGCGUUAGGAGUUUUUCUGAUAC
CAGAGUUUGUCAUGGCUUGCAACACUGAGAGUAAAACGUCAUACGUUUCAUACAUCAAU
GCAAAUCCUGGUGAGAGAGGCGGUGAGUUUACGAACACCUACAAUCCGUUAAAUACAGA
CGCCAGUGAGGGCAGAAAGUUUGCAGCAUUGGAUUAUUUGCUGGGUUCUGGUGUUCUAG
CAGGAAACGCCUUUGUGUACCCGCACCAGAUCAUCAACCUACGUACCAACAACAGUGCAA
CAAUUGUGGUGCCAUACGUAAACUCACUUGUGAUUGAUUGUAUGGCAAAACACAAUAAC
UGGGGCAUUGUCAUAUUACCACUGGCACCCUUGGCCUUUGCCGCAACAUCGUCACCACAG
GUGCCUAUUACAGUGACCAUUGCACCCAUGUGUACAGAAUUCAAUGGGUUGAGAAACAU
CACCGUCCCAGUACAUCAAGGGUUGCCGACAAUGAACACACCUGGUUCCAAUCAAUUCCU
UACAUCUGAUGACUUCCAGUCGCCCUGUGCCUUACCUAAUUUUGAUGUUACUCCACCAAU
ACACAUACCCGGGGAAGUAAAGAAUAUGAUGGAACUAGCUGAAAUUGACACAUUGAUCC
CAAUGAACGCAGUGGACGGGAAGGUGAACACAAUGGAGAUGUAUCAAAUACCAUUGAAU
GACAAUUUGAGCAAGGCACCUAUAUUCUGUUUAUCCCUAUCACCUGCUUCUGAUAAACGA
CUGAGCCGCACCAUGUUGGGUGAAAUCCUAAAUUAUUACACCCAUUGGACGGGGUCCAUC
AGGUUCACCUUUCUAUUUUGUGGUAGUAUGAUGGCCACUGGUAAACUGCUCCUCAGCUA
UUCCCCACCGGGAGCUAAACCACCAACCAAUCGCAAGGAUGCAAUGCUAGGCACACACAU
CAUCUGGGACCUAGGGUUACAAUCCAGUUGUUCCAUGGUUGCACCGUGGAUCUCCAACAC
AGUGUACAGACGGUGUGCACGUGAUGACUUCACUGAGGGCGGAUUUAUAACUUGCUUCU
AUCAAACUAGAAUUGUGGUACCUGCUUCAACCCCUACCAGUAUGUUCAUGUUAGGCUUU
GUUAGUGCGUGUCCAGACUUCAGUGUCAGACUGCUUAGGGACACUCCCCAUAUUAGUCAA
UCGAAACUAAUAGGACGUACACAAGGCAUUGAAGACCUCAUUGACACAGCGAUAAAGAA
UGCCUUAAGAGUGUCCCAACCACCCUCGACCCAGUCAACUGAAGCAACUAGUGGAGUGAA
UAGCCAGGAGGUGCCAGCUCUAACUGCUGUGGAAACAGGAGCAUCUGGUCAAGCAAUCCC
CAGUGAUGUGGUGGAAACUAGGCACGUGGUAAAUUACAAAACCAGGUCUGAAUCGUGUC
UUGAGUCAUUCUUUGGGAGAGCUGCGUGUGUCACAAUCCUAUCCUUGACCAACUCCUCCA
AGAGCGGAGAGGAGAAAAAGCAUUUCAACAUAUGGAAUAUUACAUACACCGACACUGUC
CAGUUACGCAGAAAAUUAGAGUUUUUCACGUAUUCCAGGUUUGAUCUUGAAAUGACUUU
UGUAUUCACAGAGAACUAUCCUAGUACAGCCAGUGGAGAAGUGCGAAACCAGGUGUACC
AGAUCAUGUAUAUUCCACCAGGGGCACCCCGCCCAUCAUCCUGGGAUGACUACACAUGGC
AAUCCUCUUCAAACCCUUCCAUCUUCUACAUGUAUGGAAAUGCACCUCCACGGAUGUCAA
UUCCUUACGUAGGGAUUGCCAAUGCCUAUUCACACUUCUACGAUGGCUUUGCACGGGUGC
CACUUGAGGGUGAGAACACCGAUGCUGGCGACACGUUUUACGGUUUAGUGUCCAUAAAU
GAUUUUGGAGUUUUAGCAGUUAGAGCAGUAAACCGCAGUAAUCCACAUACAAUACACAC
AUCUGUGAGAGUGUACAUGAAACCAAAACACAUUCGGUGUUGGUGCCCCAGACCUCCUCG
AGCUGUAUUAUACAGGGGAGAGGGAGUGGACAUGAUAUCCAGUGCAAUUCUACCUCUGA
CCAAGGUAGACUCAAUUACCACUUUUGGGUUUGGUCAUCAGAACAAAGCAGUGUACGUU
GCCGGUUACAAGAUUUGCAACUACCACCUAGCAACCCCAAGUGAUCACUUGAAUGCAAUU
AGUAUGUUAUGGGACAGGGAUUUAAUGGUGGUGGAAUCUAGAGCCCAGGGAACUGAUAC
CAUCGCCAGAUGUAGUUGCAGGUGUGGAGUUUACUAUUGUGAAUCUAGGAGGAAGUACU
ACCCUGUCACUUUUACUGGCCCAACGUUUCGAUUCAUGGAAGCAAACGACUACUAUCCAG
CAAGAUACCAGUCUCACAUGCUGAUAGGGUGCGGAUUUGCAGAACCCGGGGACUGCGGU
GGGAUACUGAGGUGCACUCAUGGGGUAAUUGGUAUCAUUACUGCAGGAGGUGAAGGGGU
AGUAGCCUUUGCUGACAUUAGAGACCUCUGGGUGUAUGAAGAGGAGGCCAUGGAACAGG
GAAUAACAAGCUACAUCGAAUCUCUCGGCACAGCCUUUGGCGCAGGGUUCACCCACACAA
UCAGUGAGAAAGUGACUGAAUUGACAACAAUGGUUACCAGCACUAUCACAGAAAAACUA
CUGAAAAACUUGGUGAAAAUAGUGUCGGCUCUAGUGAUUGUUGUGAGAAAUUAUGAGGA
CACUACCACGAUCCUUGCAACACUAGCACUACUCGGGUGUGAUAUAUCUCCUUGGCAAUG
GUUGAAGAAGAAGGCAUGUGACUUACUAGAGAUUCCUUAUGUGAUGCGCCAAGGUGAUG
GGUGGAUGAAGAAAUUCACAGAGGCGUGCAAUGCAGCUAAAGGCUUAGAGUGGAUUAGC
AACAAAAUUUCCAAGUUUAUAGAUUGGUUGAAGUGUAAAAUUAUCCCAGACGCUAAGGA
CAAGGUGGAAUUUCUCACCAAGUUGAAACAGCUAGACAUGUUGGAAAAUCAAAUUGCAA
CCAUCCACCAAUCUUGCCCCAGCCAAGAACAACAAGAGAUUCUUUUCAACAAUGUGAGAU
GGCUAGCAGUCCAGUCCCGUCGGUUUGCACCAUUAUACGCUGUGGAGGCACGCCGAAUUA
ACAAAAUGGAGAGCACAAUAAACAAUUAUAUACAGUUCAAGAGCAAACACCGUAUUGAA
CCAGUAUGUAUGCUCAUUCAUGGGUCACCAGGGACGGGUAAAUCUAUAGCUACUUCAUU
AAUAGGUAGAGCAAUAGCAGAGAAGGAAAGCACAUCAGUCUAUUCAAUGCCACCUGACC
CAUCUCACUUUGAUGGCUAUAAACAACAAGGGGUAGUGAUUAUGGACGACCUAAACCAA
AACCCCGAUGGUAUGGACAUGAAACUGUUUUGCCAAAUGGUAUCAACAGUGGAGUUUAU
UCCUCCAAUGGCCUCAUUAGAGGAGAAGGGCAUUUUGUUUACAUCUGAUUAUGUCCUGG
CUUCUACCAACUCUCAUUCAAUUGUACCACCCACAGUGGCUCACAGUGAUGCCUUAACCA
GACGAUUUGCAUUUGAUGUGGAGGUUUACACGAUGUCUGAACAUUCAGUCAAAGGCAAA
CUGAAUAUGGCCACGGCCACUCAAUUGUGUAAGGAUUGUCCAACACCUGCAAAUUUUAA
AAAGUGUUGCCCUCUCGUUUGUGGAAAGGCCUUGCAAUUAAUGGACAGGUACACCAGAC
AAAGGUUCACUGUAGAUGAGAUUACCACAUUAAUCAUGAAUGAGAAAAACAGAAGGGCC
AAUAUCGGCAAUUGCAUGGAAGCCUUGUUUCAAGGACCAUUAAGGUAUAAAGAUUUGAA
GAUCGAUGUGAAGACAGUUCCCCCCCCUGAGUGCAUCAGUGAUUUGUUACAAGCAGUGG
AUUCUCAAGAGGUUAGGGAUUACUGUGAGAAGAAAGGCUGGAUCGUUAACGUUACUAGC
CAGAUUCAACUAGAAAGGAACAUCAAUAGGGCCAUGACUAUACUCCAAGCUGUUACCACA
UUCGCAGCAGUCGCAGGAGUAGUGUAUGUAAUGUACAAACUCUUCGCCGGUCAACAGGG
UGCAUACACUGGCUUGCCAAACAAAAAACCCAAUGUCCCUACUAUCAGAGUCGCUAAAGU
CCAGGGGCCAGGAUUUGACUACGCAGUGGCAAUGGCAAAAAGAAACAUAGUUACUGCAA
CCACCACCAAGGGUGAAUUUACCAUGCUAGGGGUGCAUGAUAAUGUAGCAAUAUUGCCA
ACCCAUGCCGCUCCAGGAGAAACCAUUAUUAUUGAUGGGAAAGAAGUAGAGAUCCUAGA
UGCCAGAGCCUUAGAAGAUCAAGCGGGAACCAAUCUUGAGAUCACCAUUAUUACUCUAA
AAAGAAAUGAGAAGUUUAGAGACAUCAGAUCACAUAUUCCCACCCAAAUUACUGAAACU
AACGAUGGAGUGUUGAUCGUGAACACUAGCAAGUACCCCAAUAUGUAUGUCCCCGUUGG
UGCUGUGACCGAACAGGGAUAUCUUAAUCUCAGUGGACGUCAAACUGCUCGCACUUUAA
UGUACAACUUUCCAACAAGGGCAGGCCAGUGCGGAGGAAUCAUCACUUGUACUGGCAAA
GUCAUUGGGAUGCAUGUUGGCGGGAACGGUUCACAUGGGUUUGCAGCAGCCCUCAAGCG
AUCAUACUUCACUCAAAAUCAGGGCGAAAUCCAGUGGAUGAGGUCAUCAAAAGAAGUGG
GGUACCCCAUUAUAAAUGCCCCAUCCAAGACAAAGUUAGAACCCAGUGCUUUCCACUAUG
UUUUUGAAGGUGUUAAGGAACCAGCUGUACUCACUAAGAAUGACCCCAGACUAAAAACA
GAUUUUGAAGAAGCCAUCUUUUCUAAAUAUGUGGGGAACAAAAUUACUGAAGUGGACGA
GUACAUGAAAGAAGCAGUGGAUCACUAUGCAGGACAGUUAAUGUCACUGGAUAUCAACA
CAGAACAGAUGUGCCUGGAGGAUGCCAUGUACGGCACCGAUGGUCUUGAGGCCCUGGAUC
UUAGCACUAGUGCUGGAUAUCCUUAUGUUGCAAUGGGGAAAAAGAAAAGAGACAUUCUA
AAUAAACAGACCAGAGAUACUAAGGAGAUGCAGAGACUUUUAGAUACCUAUGGAAUCAA
UCUACCAUUAGUCACGUACGUGAAAGAUGAACUCAGGUCAAAGACUAAAGUGGAACAAG
GAAAGUCAAGAUUGAUUGAAGCUUCCAGCCUUAAUGAUUCAGUUGCAAUGAGAAUGGCC
UUUGGCAAUCUUUACGCAGCUUUCCACAAGAAUCCAGGUGUGGUGACAGGAUCAGCAGU
UGGUUGUGACCCAGAUUUGUUUUGGAGUAAGAUACCAGUGCUAAUGGAAGAAAAACUCU
UCGCUUUUGACUACACAGGGUAUGAUGCCUCACUCAGCCCUGCUUGGUUUGAAGCUCUUA
AAAUGGUGUUAGAAAAAAUUGGAUUUGGCAGUAGAGUAGACUAUAUAGACUACCUGAAC
CACUCUCACCACCUUUACAAAAACAAGACUUAUUGUGUCAAAGGCGGCAUGCCAUCCGGC
UGCUCUGGCACCUCAAUUUUCAACUCAAUGAUUAACAACCUGAUCAUUAGGACGCUUUUA
CUGAGAACCUACAAGGGCAUAGACUUGGACCAUUUAAAAAUGAUUGCCUAUGGUGAUGA
CGUGAUAGCUUCCUACCCCCAUGAGGUUGACGCUAGUCUCCUAGCCCAAUCAGGAAAAGA
CUAUGGACUAACCAUGACUCCAGCAGAUAAAUCAGUAACCUUUGAAACAGUCACAUGGG
AGAAUGUAACAUUUCUGAAAAGAUUUUUCAGAGCAGAUGAGAAGUAUCCAUUCCUGGUG
CAUCCAGUGAUGCCAAUGAAAGAAAUUCACGAAUCAAUCAGAUGGACCAAGGACCCUAG
AAACACACAGGAUCACGUACGCUCGUUGUGCCUAUUAGCUUGGCACAACGGUGAAGAAG
AAUACAAUAAAUUUUUAGCUAAAAUCAGAAGUGUGCCAAUUGGAAGAGCUUUAUUGCUC
CCAGAGUACUCUACAUUGUACCGCCGAUGGCUCGACUCAUUUUAGUAACCCUACCUCAGU
CGGAUUGGAUUGGGUUACACUGUUGUAGGGGUAAAUUUUUCUUUAAUUCGGAG (SEQ ID
NO: 1)

TABLE 11
Amino Acid Sequences of the 4 Viral Capsid Proteins

VP1
GIEDLIDTAIKNALRVSQPPSTQSTEATSGVNSQEVPALTAVETGASGQAIPSDVVETRHVVNYKT
RSE SCLESFFGRAACVTILSLTNSSKSGEEKKHFNIWNITYTDTVQLRRKLEFFTYSRFDLEMTFVF
TENYPSTASGEVRNQVYQIMYIPPGAPRPSSWDDYTWQSSSNPSIFYMYGNAPPRMSIPYVGIAN
AYSHFYDGFARVPLEGENTDAGDTFYGLVSINDFGVLAVRAVNRSNPHTIHTSVRVYMKPKHIR
CWCPRPPRAVLYRGEGVDMISSAILPLTKVDSITTF (SEQ ID NO: 2)
VP2
SPNVEACGYSDRVRQITLGNSTITTQEAANAIVAYGEWPTYINDSEANPVDAPTEPDVSS
NRFYTLESVSWKTTSRGWWWKLPDCLKDMGMFGQNMYYHYLGRSGYTIHVQCNASKFHQGA
LGVFLIPEFVMACNTESKTSYVSYINANPGERGGEFTNTYNPLNTDASEGRKFAALDYLLGSGVL
AGNAFVYPHQIINLRTNNSATIVVPYVNSLVIDCMAKHNNWGIVILPLAPLAFAATSSPQVPITVTI
APMCTEFNGLRNITVPVHQ (SEQ ID NO: 3)
VP3
GLPTMNTPGSNQFLTSDDFQSPCALPNFDVTPPIHIPGEVKNMMELAEIDTLIPMNAVDG
KVNTMEMYQIPLNDNLSKAPIFCLSLSPASDKRLSRTMLGEILNYYTHWTGSIRFTFLFCGSMMA
TGKLLLSYSPPGAKPPTNRKDAMLGTHIIWDLGLQSSCSMVAPWISNTVYRRCARDDFTEGGFIT
CFYQTRIVVPASTPTSMFMLGFVSACPDFSVRLLRDTPHISQSKLIGRTQ (SEQ ID NO: 4)
VP4
MGAQVSTQKTGAHENQNVAANGSTINYTTINYYKDSASNSATRQDLSQDPSKFTEPVKD
LMLKTAPALN (SEQ ID NO: 5)

### REFERENCES

1. Baggen J, Thibaut H J, Strating JRPM, van Kuppeveld FJM. (2018) The life cycle of non-polio enteroviruses and how to target it. Nature Reviews Microbiology, 16, 368-381.
2. Yin E J, Shin Y J, Kim R I, Kim T G, Chang S Y. (2017) Enterovirus 71 infection and vaccines. Clinical and Experimental Vaccine Research, 6, 4-14.
3. Pons-Salot M, Parker E P K, Grassly N C. (2015) The epidemiology of non-polio enteroviruses. Current Opinion of Infectious Diseases, 28, 479-487.
4. Kaufman H L, Kohlhapp F J, Zloza A. (2015) Oncolytic viruses: a new class of immunotherapy drugs. Nature Reviews Cancer Immunotherapy, 14, 642-662.
5. Bradley S, Jakes A D, Harrington K, Pandha H, Melcher A, Erringon-Mais F. (2014). Applications of coxsackievirus A21 in oncology. Oncolytic Virotherapy, 3, 47-55.
6. Shingler K L, Organtini L J, Hafenstein S. (2016). Enterovirus 71 virus propagation and purification. Bio Protocols, 4(9), e1117.
7. Tomono T, Hirai Y, Okada H, Adachie K, Ishii A, Shimada T, Onodera M, Tamaoka A, Okada T. (2016) Ultracentrifugation-free chromatography-mediated large-scale purification of recombinant adeno-associated virus serotype 1 (rAAV1). Molecular Therapy. Methods & Clinical Development, 3, 15058.
8. Pompella A, Visvikis A, Paolicchi D, De Tata V, Casini A F. (2003). The changing faces of glutathione, a cellular protagonist. Biochemical Pharmacology, 66, 1499-1503.
9. Pina A S, Lowe C R, Roque A C A. (2014) Challenges and opportunities in the purification of recombinant tagged proteins. Biotechnology Advances, 32(2), 366-381.
10. Jiang P, Liu Y, Ma H C, Paul A V, Wimmer E. (2014) Picornavirus Morphogenesis. Microbiology and Molecular Biology Reviews, 78(3), 418-437.
11. Dalldorf et al., 1949 (J. exp. Med. 89: 567)
12. Lennette et al., 1958 (Am. J. Hyg. 68: 272)
13. Mirkovic et al., 1974 (Intervirol. 4: 119-127)
14. Sickles et al., 1959 (Proc. Soc. exp. Biol. Med. 102: 742-743)
15. Abraham and Cheever, 1963. (PSEBM 112: 981)
16. Loughney, J. W., Lancaster, C., Ha, S., & Rustandi, R. R. (2014). Residual bovine serum albumin (BSA) quantitation in vaccines using automated Capillary Western technology. Analytical biochemistry, 461, 49-56.
17. Konstantinidis, S., Gob, H. Y., Martin Bufájer, J. M., de Galbert P., Parau. M., & Velayudhan. A. (2018). Flexible and accessible automated operation of miniature chromatography columns on a liquid handling station. Biotechnology Journal, 13(3), 1700390.
18. Klasse, P. J. (2015). Molecular determinants of the ratio of inert to infectious virus particles. Progress in molecular biology and translational science, 129, 285-326.
19. Koonin E V. The phylogeny of RNA-dependent RNA polymerases of positive-strand RNA viruses. J Gen Virol. 1991; 72:2197-2206.
20. Stewart S R, Semler B L. RNA determinants of picornavirus Cap-independent translation initiation. Semin Virol. 1997; 8:242-255.
21. Xiao C et al. The crystal structure of Coxsackievirus A21 and its interaction with ICAM-1. Structure. 2005; 13:1019-1033.
22. Dougherty R M. Animal virus titration techniques. Techniques in experimental; virology. 1964; 178.

U.S. provisional patent application No. 62/951,078 is incorporated herein by reference in its entirety. All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

What is claimed:

1. A composition comprising purified Coxsackievirus A 21 (CVA21) comprising VP0, VP1, VP2, VP3, VP4 and CVA21 RNA, wherein the VP0 to VP2 ratio is less than about 0.01.

2. The composition of claim 1, wherein the VP0 to VP2 ratio is about 0.0005-0.005.

3. The composition of claim 1, wherein the VP0 to VP2 ratio is about 0.001-0.003.

4. A composition comprising purified CVA21, wherein the genome to infectivity ratio is less than about 5000 genome/pfu.

5. The composition of claim 4, wherein the genome to infectivity ratio is about 200-2000 genome/pfu.

6. The composition of claim 4, wherein the genome to infectivity ratio is about 200-800 genome/pfu.

7. A composition comprising purified CVA21, wherein the particle to infectivity ratio is less than about 5000 particle/pfu.

8. The composition of claim 7, wherein the particle to infectivity ratio is about 200-2000 particle/pfu.

9. The composition of claim 7, wherein the particle to infectivity ratio is about 200-600 particle/pfu.

10. The composition of claim 6, wherein the total VP1+VP2+VP3+VP4 peak area/total peak area is at least 95%.

11. The composition of claim 4, wherein the amount of host cell DNA in the composition is less than about 10,000 pg/dose, with about 5E7 pfu CVA21 per dose.

12. The composition of claim 9, wherein the amount of host cell DNA in the composition is about 0.05-10 pg/dose, with about 5E7 pfu CVA21 per dose.

13. The composition of claim 4, wherein the amount of bovine serum albumin in the composition is less than about 50,000 pg/dose, with about 5E7 pfu CVA21 per dose.

14. The composition of claim 9, wherein the amount of bovine serum albumin in the composition is about 50-150 pg/dose, with about 5E7 pfu CVA21 per dose.

15. The composition of claim 1, wherein the CVA21 comprises the nucleotide sequence in SEQ ID NO: 1.

16. The composition of claim 1, wherein the composition has a potency of 1E5 to 1E12 TCID₅₀/ml or pfu/ml.

17. The composition of claim 4, wherein the composition has a potency of 1E5 to 1E12 TCID₅₀/ml or pfu/ml.

18. The composition of claim 7, wherein the composition has a potency of 1E5 to 1E12 TCID₅₀/ml or pfu/ml.

19. A pharmaceutical composition comprising the composition of claim 1 and a pharmaceutically acceptable excipient.

20. A method of treating cancer in a patient comprising intratumorally administering to the patient a dose of up to about 3E8 TCID₅₀ or 5E7 pfu per treatment of the pharmaceutical composition of claim 19.

21. The method of claim 20, wherein the dose is about 3E7 to about 3E8 TCID₅₀ or about 5E6 to about 5E7 pfu per treatment of the pharmaceutical composition.

22. A method of treating cancer in a patient comprising intravenously administering to the patient a dose of about 1E9 TCID₅₀ or 1.5E8 pfu per treatment of the pharmaceutical composition of claim 19.

23. A method of purifying an enterovirus comprising the steps of:
(a)binding the enterovirus to a stationary phase using a loading solution, wherein glutathione is immobilized to the stationary phase;
(b)eluting the enterovirus from the stationary phase with an elution solution.

24. The method of claim 23, wherein prior to step (a), the stationary phase is equilibrated with an equilibration solution.

25. The method of claim 24, further comprising step (i) of washing the stationary phase with one or more wash solutions after step (a) and prior to step (b).

26. The method of claim 25, wherein step (i) comprises a first wash step with a wash solution having a conductivity higher than the equilibration solution or loading solution.

29. The method of claim 26, wherein step (i) comprises a second wash step with a wash solution having a conductivity lower than the wash solution in the first wash step.

28. The method of claim 27, wherein the conductivity of the elution solution is the same as the wash solution in the second wash step.

29. The method of claim 25, wherein one or more of the loading solution, equilibration solution, the one or more wash solutions and the elution solution has a pH of about 5-10.

30. The method of claim 25, wherein one or more of the loading solution, equilibration solution, the one or more wash solutions and the elution solution has a pH of about 6-9.

31. The method of claim 29, wherein one or more of the loading solution, equilibration solution, the one or more wash solutions and the elution solution further comprises a surfactant.

32. The method of claim 31, wherein the surfactant is PS-80 or PS-20.

33. The method of claim 31, wherein the surfactant is about 0.001-1% w/v PS-80.

34. The method of claim 31, wherein the surfactant is about 0.001-0.1% w/v PS-80.

35. The method of claim 24, wherein the loading solution or equilibration solution comprises about 50-200 mM monovalent salt.

36. The method of claim 25, wherein the one or more wash solutions comprises about 50-500 mM monovalent salt.

37. The method of claim 25, wherein the first wash solution comprises about 350-500 mM NaCl or KCl and the second wash solution comprises about 50-200 mM NaCl or KCl.

38. The method of claim 23, wherein the elution solution comprises about 50-500 mM of monovalent salt.

39. The method of claim 23, wherein the elution solution comprises about 50-200 mM of NaCl or KCl.

40. The method of claim 23, wherein the elution solution comprises about 0.1-100 mM glutathione.

41. The method of claim 23, wherein the elution solution comprises about 0.1-25 mM glutathione.

42. The method of claims 23, wherein the elution solution comprises about 0.5-5 mM glutathione and 75-150 mM NaCl or KCl.

43. The method of claim 25, wherein the wash or elution solution further comprises one or more of EDTA, DTT and 2-mercaptoethanol.

44. A method of purifying an enterovirus comprising the steps of:
(c)loading the enterovirus to an anionic exchange column using a loading solution,
(d)collecting the enterovirus from the flow-through.

45. The method of claim 44, wherein the loading solution comprises about 50-500 mM monovalent salt concentration at pH about 6-9.

46. The method of claim 23, wherein full mature enterovirus is purified.

47. The method of claim 23, wherein the enterovirus is a Group B or C enterovirus.

48. The method of claim 23, wherein the enterovirus is Echovirus, Rhinovirus A, B or C.

49. The method of claim 23, wherein the enterovirus is Echovirus 1, Rhinovirus 1B, Rhinovirus 35, Coxsackievirus A 13 (CVA13), Coxsackievirus A 15 (CVA15), Coxsackievirus A 18 (CVA18), Coxsackievirus A 20 (CVA20), or Coxsackievirus A 21 (CVA21).

50. The method of claim 23, wherein the enterovirus is CVA1, CVA11, CVA13, CVA15, CVA17, CVA18, CVA19, CVA20a, CVA20b, CVA20c, CVA21, CVA22 or CVA24.

51. The method of claim 50, wherein the enterovirus is CVA21.

52. A purified composition of CVA21 produced by the method of claim 51.
